(19) United States
(12) Patent Application Publication
(10) Pub. No.: US 2024/0002812 A1
(43) Pub. Date: Jan. 4, 2024

(54) METHODS OF PREPARING GENE THERAPIES

(71) Applicant: BridgeBio Services Inc., Palo Alto, CA (US)

(72) Inventors: Ashton LAVOIE, Palo Alto, CA (US); Jeffrey Zugates, Palo Alto, CA (US)

(21) Appl. No.: 18/319,350

(22) Filed: May 17, 2023

Related U.S. Application Data
(60) Provisional application No. 63/342,961, filed on May 17, 2022.

(51) Int. Cl. C12N 7/00; C12N 15/86; G01N 30/02

(52) U.S. Cl. CPC C12N 7/00; C12N 15/86; G01N 30/02; C12N 2750/14151; G01N 2030/027

(57) ABSTRACT

The disclosure provides methods for purifying gene therapy compositions using anion exchange chromatography.

### CROSS REFERENCE TO RELATED APPLICATIONS

The present Application claims the benefit of priority to U.S. Provisional Application No. 63/342,961, filed on May 17, 2022, the contents of which are hereby incorporated by reference in their entirety for all purposes.

### BACKGROUND

Gene therapies are promising methods for the treatment of various diseases. Gene therapies generally involve introduction of a nucleic acid molecule directly to a patient or into cells that are subsequently provided to a patient. Viral based systems including adeno-associated virus (AAV)-based systems have been extensively explored for use as gene delivery vectors for gene therapies. AAV is not associated with any known human or animal disease and does not appear to have deleterious health effects. Various AAV vectors (including recombinant AAV vectors (rAAV vectors)) can be engineered to include a nucleic acid sequence of interest, such as a transgene, antisense molecule, or other nucleic acid molecule capable of having or leading to a beneficial therapeutic effect. AAV therapies have shown promise in clinical trials across several indications.

Questions regarding the safety and efficacy of AAV therapies remain unresolved. Production of AAV therapeutics generally involves an upstream phase in which AAV vectors are produced; a downstream phase in which AAV vectors are purified from certain impurities; and a formulation phase, in which the AAV vectors are prepared for dosing in patients. Because of the complex steps involved in the production of rAAV vectors, a range of impurities including proteins, nucleic acids, process additives, and vector-related impurities such as aggregated vectors and empty capsids may arise. Such impurities can arise during, e.g., transfection of plasmid DNA into cells to produce rAAV vectors, as well as during lysis of cells to release rAAV vectors from cells. Empty capsids (e.g., rAAVs that do not include a nucleic acid molecule of therapeutic interest) and aggregated vectors may have various negative effects including reduction of transduction efficiency, induction of undesirable antibody responses, and potential contribution to destruction of transduced cells. The range of potential impurities present significant challenges to commercial scale up of gene therapies.

Various methods of purifying rAAV vectors of interest have been explored, but improving yield and product quality remain challenging. Accordingly, there is a need for improved methods of purifying gene therapies.

### SUMMARY

The present disclosure provides methods for processing and purifying gene therapy compositions. The methods provided herein may be useful in separating adeno-associated (AAV) (e.g., recombinant AAV (rAAV)) vector particles including a therapeutic nucleic acid molecule or other payload of interest from AAV vector particles that do not include the therapeutic nucleic acid molecule or other payload of interest. The methods may comprise ion exchange chromatographic methods, such as anion exchange chromatography.

In an aspect, the present disclosure provides a method, comprising: a) providing a mixture comprising a plurality of recombinant adeno-associated (rAAV) vector particles and one or more impurities, wherein rAAV vector particles of the plurality of rAAV vector particles each include a therapeutic nucleic acid molecule, and wherein the one or more impurities comprise rAAV vector particle aggregates, rAAV vector particles that do not include the therapeutic nucleic acid molecule, and aggregates of rAAV vector particles and rAAV vector particles that do not include the therapeutic nucleic acid molecule; b) contacting an anion exchange column with the mixture; and c) contacting the anion exchange column with a first elution buffer and a second elution buffer to provide an eluate, wherein the eluate comprises rAAV vector particles of the plurality of rAAV vector particles. In some embodiments, the first elution buffer comprises a first salt (e.g., a sulfate salt, such as magnesium sulfate) and the second elution buffer comprises a second salt (e.g., a sulfate salt, such as magnesium sulfate) and a third salt (e.g., a chloride salt, such as magnesium chloride). In some embodiments, the total salt concentration of the second elution buffer is between about 40-250 millimolar (mM).

Additional aspects and embodiments are described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an overview of an rAAV vector particle production process.

FIG. 2 shows an anion exchange chromatogram of an AAV5 vector particle produced using a linear sodium chloride gradient across a monolithic chromatography column functionalized with a quaternary amine and showing a ˜3× enrichment factor. The capsid has a low vector genome (vg) yield range (˜47%±16%), poor robustness/reproducibility in chromatographic appearance and yield, and a lack of flexibility in available column sizing/required column cycling.

FIG. 3 shows the chromatogram corresponding to a single salt linear sodium chloride gradient across a packed bed quaternary amine chromatography column. Poor separation between the empty capsid peak (peak 1), the full capsid peak (peak 2), and the impurity peak (peak 3) is observed using this single salt sodium chloride gradient.

FIG. 4 shows chromatograms for a single salt linear gradient screening.

FIG. 5 shows an analysis of the single salt screening.

FIG. 6 shows chromatograms for the dual salt linear gradient screening.

FIG. 7 shows chromatograms from a second dual salt linear gradient screening when low ionic strength magnesium salts are introduced in the first elution buffer.

FIG. 8 shows chromatograms from a dual salt gradient screening performed using linear, step, linear with hold, and hybrid gradient schemes.

FIGS. 9A-9F depict AEX elution chromatograms for single salt linear gradient screening. AEX chromatographic profiles during elution with varied salt gradients. Gradients included the following combinations over 100 CV: 0-200 mM sodium chloride (FIG. 9A), 0-200 mM sodium acetate (FIG. 9B), 0-120 mM sodium sulfate (FIG. 9C), 0-50 mM calcium chloride (FIG. 9D), 0-50 mM magnesium chloride (FIG. 9E), 0-37.5 mM magnesium sulfate (FIG. 9F).

FIGS. 10A-10G depict AEX elution chromatograms for dual salt linear gradient screening. AEX chromatographic profiles during elution with varied salt gradients. Gradients included the following combinations over 100 CV: 0-33 mM sodium sulfate and 0-33 mM magnesium chloride (FIG. 10A), 0-25 mM magnesium sulfate and 0-33 mM sodium sulfate (FIG. 10B), 0-25 mM magnesium sulfate and 0-100 mM sodium acetate (FIG. 10C), 0-33 mM magnesium chloride and 0-100 mM sodium acetate (FIG. 10D), 0-33 mM magnesium chloride and 0-100 mM sodium chloride (FIG. 10E), 0-25 mM magnesium sulfate and 0-100 mM sodium chloride (FIG. 10F), and 0-25 mM magnesium sulfate and 0-33 mM magnesium chloride (FIG. 10G).

FIGS. 11A-11E depict AEX elution chromatograms for dual salt linear gradients with initial low concentration magnesium salt. AEX chromatographic profiles during elution with varied salt gradients, and initial magnesium salt concentrations, as depicted in Table 4. Gradients included the following combinations over 100 CV: 2-33 mM magnesium chloride and 0-25 mM magnesium sulfate (FIG. 11A), 1.5-25 mM magnesium sulfate and 0-33 mM magnesium chloride (FIG. 11B), 1.5-25 mM magnesium sulfate and 0-100 mM sodium acetate (FIG. 11C), 2-33 mM magnesium chloride and 0-100 mM sodium acetate (FIG. 11D), and 1.5-25 mM magnesium sulfate and 0-33 mM sodium sulfate (FIG. 11E).

FIGS. 12A-12B depict AEX elution chromatograms for optimized AEX compared to initial process. Overlayed AEX chromatographic profiles during elution for optimized conditions shown are representative of: Run F and Run G (FIG. 12A), and Run I and Run J (FIG. 12B).

### DETAILED DESCRIPTION

The present disclosure provides methods of processing compositions for use in gene therapies. The methods provided herein may be used to separate empty and/or aggregated adeno-associated (AAV) particles (e.g., recombinant AAV (rAAV) particles) from AAV particles including a therapeutic nucleic acid molecule of interest. The methods provided herein may be used to purify AAV particles including a therapeutic nucleic acid molecule of interest. The methods provided herein comprise the use of anion exchange chromatography methods, which methods may comprise the use of one or more buffer solutions comprising two or more salts.

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

The terms “a”, “an”, and “the” refer to “one or more” when used in this application, including the claims. Thus, for example, reference to “a carrier” includes mixtures of one or more carriers, two or more carriers, and the like and reference to “the method” includes reference to equivalent steps and/or methods known to those skilled in the art, and so forth.

In the present description, any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 0% to 10%.

Also as used herein, “and/or” refers to and encompasses any and all possible combinations of one or more of the associated listed items, as well as the lack of combinations when interpreted in the alternative (“or”). The use of the alternative (e.g., “or”) should be understood to mean either one, both, or any combination thereof of the alternatives.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like.

The term “pharmaceutically acceptable”, unless otherwise noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

As used herein, “treatment,” “treating,” “palliating,” and “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); and (3) relieving the condition (for example, by causing regression, or reducing the severity of the state, disorder or condition or at least one of its clinical or subclinical symptoms). For example, beneficial clinical results include, but are not limited to, delay or slowing of invasiveness or growth of tumors or hamartomas, and amelioration of symptoms associated with such tumors or hamartomas. Treatment also includes a decrease in mortality or an increase in the lifespan of a subject as compared to one not receiving the treatment.

The term “effective amount” or “therapeutically effective amount” refers to the amount of an agent that is sufficient to achieve an outcome, for example, to effect beneficial or desired results, such as, treatment of tuberous sclerosis or a symptom thereof. The therapeutically effective amount may vary depending upon one or more of: the subject and disease condition being treated, the weight and age of the subject, the severity of the disease condition, the manner of administration, and the like. A therapeutically effective amount may be an amount sufficient to treat tuberous sclerosis and/or to ameliorate, diminish the severity of, eliminate, and/or delay the onset of one or more symptoms of tuberous sclerosis.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. In some embodiments, the subject is a human. A human subject may be an adult, a teenager (e.g., 12 years to 18 years of age), a child (e.g., 2 years to 14 years of age), an infant (e.g., 1 month to 24 months old), or a neonate (up to 1 month old). In some embodiments, an adult is a senior about 60 years or older, such as about 65 years or older. In some embodiments, the subject is a pregnant woman or a woman intending to become pregnant. In some embodiments, the subject is less than 18 years of age.

An “adeno-associated virus (AAV) expression cassette” is a nucleic acid that gets packaged into a recombinant AAV vector, and comprises a sequence encoding one or more transgenes flanked by a 5′ inverted terminal repeat (ITR) and a 3′ITR.

As used herein, the terms “virus vector,” “viral vector,” and “gene delivery vector” refer to a virus particle that functions as a nucleic acid delivery vehicle, and which comprises a nucleic acid molecule (e.g., an AAV expression cassette) packaged within a virion. Exemplary virus vectors include adeno-associated virus vectors (AAVs).

As used herein, the term “adeno-associated virus” (AAV), includes but is not limited to, AAV type 1 (e.g., AAV of serotype 1, also referred to as AAV1), AAV type 2 (e.g., AAV2), AAV type 3 (e.g., AAV3, including types 3A and 3B, AAV3A and AAV3B), AAV type 4 (e.g., AAV4), AAV type 5 (e.g., AAV5), AAV type 6 (e.g., AAV6), AAV type 7 (e.g., AAV7), AAV type 8 (e.g., AAV8), AAV type 9 (e.g., AAV9), AAV type 10 (e.g., AAV10), AAV type 11 (e.g., AAV11), AAV type 12 (e.g., AAV12), AAV type 13 (e.g., AAV13), AAV type rh32.33 (e.g., AAVrh32.33), AAV type rh8 (e.g., AAVrh8), AAV type rh10 (e.g., AAVrh10), AAV type rh74 (e.g., AAVrh74), AAV type hu.68 (e.g., AAVhu.68), avian AAV (e.g., AAAV), bovine AAV (e.g., BAAV), canine AAV, equine AAV, ovine AAV, snake AAV, bearded dragon AAV, AAV2i8, AAV2g9, AAV-LK03, AAV7m8, AAV Anc80, AAV PHP.B, and any other AAV now known or later discovered.

As used herein “sequence identity” refers to the extent to which two optimally aligned polynucleotides or polypeptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. “Percent identity” is the identity fraction times 100. The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539. For the purposes of calculating identity to a sequence, extensions such as tags are not included.

As used herein, a nucleic acid sequence (e.g., coding sequence) and regulatory sequences are the to be “operably linked” when they are covalently linked in such a way as to place the expression or transcription of the nucleic acid sequence under the influence or control of the regulatory sequences. If it is desired that the nucleic acid sequences be translated into a functional protein, two DNA sequences are the to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein.

As used herein, “codon optimization” refers to modifying a nucleic acid sequence to change individual nucleic acids without any resulting change in the corresponding encoded amino acid. Sequences modified in this way are referred to herein as “codon optimized.” Methods of performing codon optimization are described in U.S. Pat. Nos. 7,561,972, 7,561,973, and 7,888,112, each of which is incorporated herein by reference in their entireties for all purposes. In some embodiments, the sequence surrounding the translational start site can be converted to a consensus Kozak sequence as described further in Kozak et al., Nucleic Acids Res. 15(20):8125-81 48 (1987), incorporated herein by reference in its entirety for all purposes.

### Methods of Processing Gene Therapies

The methods provided herein may be used to process compositions for use in gene therapy, such as compositions including a mixture of recombinant adeno-associated virus (rAAV) particles that each comprise a therapeutic nucleic acid molecule of interest and various impurities. Impurities in such a mixture may include, for example, rAAV particles that do not include the therapeutic nucleic acid molecule of interest, such as unfilled (e.g., “empty”) rAAV particles and rAAV particles including other nucleic acid molecules, as well as aggregates of various rAAV particles (e.g., aggregates of rAAV particles including the therapeutic nucleic acid molecule of interest, aggregates of empty rAAV particles, aggregates of empty rAAV particles and rAAV particles including the therapeutic nucleic acid molecule of interest, etc.). The methods provided herein include anion exchange chromatographic methods, such as anion exchange chromatographic methods comprising the use of an elution buffer including two or more salts.

Accordingly, in an aspect, the present disclosure provides a method, comprising: a) providing a mixture comprising a plurality of recombinant adeno-associated (rAAV) vector particles and one or more impurities, wherein rAAV vector particles of the plurality of rAAV vector particles each include a therapeutic nucleic acid molecule, and wherein the one or more impurities comprise rAAV vector particle aggregates, rAAV vector particles that do not include the therapeutic nucleic acid molecule, and aggregates of rAAV vector particles and rAAV vector particles that do not include the therapeutic nucleic acid molecule; b) contacting an anion exchange column with the mixture; and c) contacting the anion exchange column with a first elution buffer and a second elution buffer to provide an eluate, wherein the eluate comprises rAAV vector particles of the plurality of rAAV vector particles, wherein the first elution buffer comprises magnesium sulfate; and wherein the second elution buffer comprises magnesium sulfate and magnesium chloride.

In some embodiments, the first elution buffer comprises between about 0 millimolar (mM) to about 10 mM magnesium sulfate. In some embodiments, the first elution buffer comprises about 2 mM magnesium sulfate. In some embodiments, the second elution buffer comprises between about 10 millimolar (mM) to about 50 mM magnesium sulfate. In some embodiments, the second elution buffer comprises between about 10 millimolar (mM) to about 40 mM magnesium sulfate. In some embodiments, the second elution buffer comprises between about 10 millimolar (mM) to about 30 mM magnesium sulfate. In some embodiments, the second elution buffer comprises between about 30 mM to about 40 mM magnesium sulfate. In some embodiments, the second elution buffer comprises between about 10 millimolar (mM) to about 50 mM magnesium chloride. In some embodiments, the second elution buffer comprises between about 10 millimolar (mM) to about 30 mM magnesium chloride. In some embodiments, the second elution buffer comprises between about 20 mM to about 30 mM magnesium chloride.

In some embodiments, the total salt concentration of the first elution buffer and/or second elution buffer is between about 0-500 millimolar (mM), such as between about 0-400 mM, between about 0-300 mM, between about 0-250 mM, between about 0-200 mM, between about 0-150 mM, between about 0-100 mM, between about 0-90 mM, between about 0-80 mM, between about 0-70 mM, between about 0-60 mM, between about 0-50 mM, between about 0-40 mM, between about 0-30 mM, between about 0-20 mM, between about 0-10 mM, between about 10-300 mM, between about 10-250 mM, between about 10-200 mM, between about 10-150 mM, between about 10-100 mM, between about 10-90 mM, between about 10-80 mM, between about 10-70 mM, between about 10-60 mM, between about 10-50 mM, between about 10-40 mM, between about 20-300 mM, between about 20-250 mM, between about 20-200 mM, between about 20-150 mM, between about 20-100 mM, between about 20-90 mM, between about 20-80 mM, between about 20-70 mM, between about 20-60 mM, between about 20-50 mM, between about 20-40 mM, between about 30-300 mM, between about 30-250 mM, between about 30-200 mM, between about 30-150 mM, between about 30-100 mM, between about 30-90 mM, between about 30-80 mM, between about 30-70 mM, between about 30-60 mM, between about 30-50 mM, between about 30-40 mM, between about 40-300 mM, between about 40-250 mM, between about 40-200 mM, between about 40-150 mM, between about 40-100 mM, between about 40-90 mM, between about 40-80 mM, between about 40-70 mM, between about 40-60 mM, between about 40-50 mM, between about 50-300 mM, between about 50-250 mM, between about 50-200 mM, between about 50-150 mM, between about 50-100 mM, between about 50-90 mM, between about 50-80 mM, between about 50-70 mM, between about 50-60 mM, between about 60-300 mM, between about 60-250 mM, between about 60-200 mM, between about 60-150 mM, between about 60-100 mM, between about 60-90 mM, between about 60-80 mM, between about 60-70 mM, between about 70-300 mM, between about 70-250 mM, between about 70-200 mM, between about 70-150 mM, between about 70-100 mM, between about 70-90 mM, between about 70-80 mM, between about 80-300 mM, between about 80-250 mM, between about 80-200 mM, between about 80-150 mM, between about 80-100 mM, between about 80-90 mM, between about 90-300 mM, between about 90-250 mM, between about 90-200 mM, between about 90-150 mM, between about 90-100 mM, between about 100-300 mM, between about 100-250 mM, between about 100-200 mM, between about 100-150 mM, between about 150-300 mM, between about 150-250 mM, between about 150-200 mM, between about 200-300 mM, between about 200-250 mM, between about 250-300 mM, or any range therein.

In some embodiments, the total salt concentration of the first elution buffer is between about 0-300 mM, between about 0-250 mM, between about 0-200 mM, between about 0-150 mM, between about 0-100 mM, between about 0-90 mM, between about 0-80 mM, between about 0-70 mM, between about 0-60 mM, between about 0-50 mM, between about 0-40 mM, between about 0-30 mM, between about 0-20 mM, or between about 0-10 mM. In some embodiments, the total salt concentration of the first elution buffer is between about 0-10 mM, between about 0-5 mM, or between about 0-3 mM.

In some embodiments, the total salt concentration of the second elution buffer is between about 0-500 millimolar (mM), such as between about 0-400 mM, between about 0-300 mM, between about 0-250 mM, between about 0-200 mM, between about 0-150 mM, between about 0-100 mM, between about 0-90 mM, between about 0-80 mM, between about 0-70 mM, between about 0-60 mM, between about 0-50 mM, between about 0-40 mM, between about 0-30 mM, between about 0-20 mM, between about 0-10 mM, between about 10-300 mM, between about 10-250 mM, between about 10-200 mM, between about 10-150 mM, between about 10-100 mM, between about 10-90 mM, between about 10-80 mM, between about 10-70 mM, between about 10-60 mM, between about 10-50 mM, between about 10-40 mM, between about 20-300 mM, between about 20-250 mM, between about 20-200 mM, between about 20-150 mM, between about 20-100 mM, between about 20-90 mM, between about 20-80 mM, between about 20-70 mM, between about 20-60 mM, between about 20-50 mM, between about 20-40 mM, between about 30-300 mM, between about 30-250 mM, between about 30-200 mM, between about 30-150 mM, between about 30-100 mM, between about 30-90 mM, between about 30-80 mM, between about 30-70 mM, between about 30-60 mM, between about 30-50 mM, between about 30-40 mM, between about 40-300 mM, between about 40-250 mM, between about 40-200 mM, between about 40-150 mM, between about 40-100 mM, between about 40-90 mM, between about 40-80 mM, between about 40-70 mM, between about 40-60 mM, between about 40-50 mM, between about 50-300 mM, between about 50-250 mM, between about 50-200 mM, between about 50-150 mM, between about 50-100 mM, between about 50-90 mM, between about 50-80 mM, between about 50-70 mM, between about 50-60 mM, between about 60-300 mM, between about 60-250 mM, between about 60-200 mM, between about 60-150 mM, between about 60-100 mM, between about 60-90 mM, between about 60-80 mM, between about 60-70 mM, between about 70-300 mM, between about 70-250 mM, between about 70-200 mM, between about 70-150 mM, between about 70-100 mM, between about 70-90 mM, between about 70-80 mM, between about 80-300 mM, between about 80-250 mM, between about 80-200 mM, between about 80-150 mM, between about 80-100 mM, between about 80-90 mM, between about 90-300 mM, between about 90-250 mM, between about 90-200 mM, between about 90-150 mM, between about 90-100 mM, between about 100-300 mM, between about 100-250 mM, between about 100-200 mM, between about 100-150 mM, between about 150-300 mM, between about 150-250 mM, between about 150-200 mM, between about 200-300 mM, between about 200-250 mM, between about 250-300 mM, or any range therein. In some embodiments, the total salt concentration of the second elution buffer is between about 40-80 millimolar (mM), such as between about 40-50 mM, between about 50-60 mM, between about 60-70 mM, or between about 70-80 mM.

In a related aspect, the present disclosure provides a method, comprising: a) providing a mixture comprising a plurality of recombinant adeno-associated (rAAV) vector particles and one or more impurities, wherein rAAV vector particles of the plurality of rAAV vector particles each include a therapeutic nucleic acid molecule, and wherein the one or more impurities comprise rAAV vector particle aggregates, rAAV vector particles that do not include the therapeutic nucleic acid molecule, and aggregates of rAAV vector particles and rAAV vector particles that do not include the therapeutic nucleic acid molecule; b) contacting an anion exchange column with the mixture; and c) contacting the anion exchange column with a first elution buffer and a second elution buffer to provide an eluate, wherein the eluate comprises rAAV vector particles of the plurality of rAAV vector particles, wherein the first elution buffer comprises a sulfate salt; and wherein the second elution buffer comprises a sulfate salt and a chloride salt.

In some embodiments, the first elution buffer comprises magnesium sulfate. In some embodiments, the first elution buffer comprises between about 0 millimolar (mM) to about 10 mM magnesium sulfate. In some embodiments, the first elution buffer comprises about 2 mM magnesium sulfate. In some embodiments, the second elution buffer comprises magnesium sulfate. In some embodiments, the second elution buffer comprises between about 10 millimolar (mM) to about 50 mM magnesium sulfate. In some embodiments, the second elution buffer comprises between about 30 mM to about 40 mM magnesium sulfate. In some embodiments, the second elution buffer comprises magnesium chloride. In some embodiments, the second elution buffer comprises between about 10 millimolar (mM) to about 50 mM magnesium chloride. In some embodiments, the second elution buffer comprises between about 20 mM to about 30 mM magnesium chloride.

In some embodiments, the total salt concentration of the first elution buffer and/or second elution buffer is between about 0-500 millimolar (mM), such as between about 0-400 mM, between about 0-300 mM, between about 0-200 mM, between about 0-100 mM, between about 0-90 mM, between about 0-80 mM, between about 0-70 mM, between about 0-60 mM, between about 0-50 mM, between about 0-40 mM, between about 0-30 mM, between about 0-20 mM, between about 0-10 mM, between about 10-100 mM, between about 10-90 mM, between about 10-80 mM, between about 10-70 mM, between about 10-60 mM, between about 10-50 mM, between about 10-40 mM, between about 20-100 mM, between about 20-90 mM, between about 20-80 mM, between about 20-70 mM, between about 20-60 mM, between about 20-50 mM, between about 20-40 mM, or any range therein.

In some embodiments, the total salt concentration of the first elution buffer is between about 0-100 mM, between about 0-90 mM, between about 0-80 mM, between about 0-70 mM, between about 0-60 mM, between about 0-50 mM, between about 0-40 mM, between about 0-30 mM, between about 0-20 mM, or between about 0-10 mM. In some embodiments, the total salt concentration of the first elution buffer is between about 0-10 mM, between about 0-5 mM, or between about 0-3 mM.

In some embodiments, the total salt concentration of the second elution buffer is between about 40-80 millimolar (mM), such as between about 40-50 mM, between about 50-60 mM, between about 60-70 mM, or between about 70-80 mM.

In a related aspect, the present disclosure provides a method, comprising: a) providing a mixture comprising a plurality of recombinant adeno-associated (rAAV) vector particles and one or more impurities, wherein rAAV vector particles of the plurality of rAAV vector particles each include a therapeutic nucleic acid molecule, and wherein the one or more impurities comprise rAAV vector particle aggregates, rAAV vector particles that do not include the therapeutic nucleic acid molecule, and aggregates of rAAV vector particles and rAAV vector particles that do not include the therapeutic nucleic acid molecule; b) contacting an anion exchange column with the mixture; and c) contacting the anion exchange column with a first elution buffer and a second elution buffer to provide an eluate, wherein the eluate comprises rAAV vector particles of the plurality of rAAV vector particles, wherein the first elution buffer comprises a first salt; and wherein the second elution buffer comprises a second salt and a third salt, wherein the total salt concentration of the second elution buffer is between about 40-80 millimolar (mM).

In some embodiments, the first salt and the second salt are of the same type. In some embodiments, the first salt and the third salt are of different types. In some embodiments, the first, second, and third salts are selected from the group consisting of a sulfate salt, a chloride salt, and an acetate salt. In some embodiments, the first salt and the second salt are sulfate salts. In some embodiments, the first salt and the second salt are magnesium sulfate. In some embodiments, the third salt is a chloride salt. In some embodiments, the third salt is a magnesium chloride.

In some embodiments, the first elution buffer comprises magnesium sulfate. In some embodiments, the first elution buffer comprises between about 0 millimolar (mM) to about 10 mM magnesium sulfate. In some embodiments, the first elution buffer comprises about 2 mM magnesium sulfate. In some embodiments, the second elution buffer comprises magnesium sulfate. In some embodiments, the second elution buffer comprises between about 10 millimolar (mM) to about 50 mM magnesium sulfate. In some embodiments, the second elution buffer comprises between about 30 mM to about 40 mM magnesium sulfate. In some embodiments, the second elution buffer comprises magnesium chloride. In some embodiments, the second elution buffer comprises between about 10 millimolar (mM) to about 50 mM magnesium chloride. In some embodiments, the second elution buffer comprises between about 20 mM to about 30 mM magnesium chloride.

In some embodiments of any of the preceding aspects, the first elution buffer and/or the second elution buffer further comprises bis-tris propane. In some embodiments, the first elution buffer comprises bis-tris propane. In some embodiments, the second elution buffer comprises bis-tris propane. In some embodiments, the first elution buffer and/or the second elution buffer comprises 10-30 mM bis-tris propane.

In some embodiments of any of the preceding aspects, the first elution buffer and/or the second elution buffer further comprises a poloxamer. In some embodiments, the first elution buffer comprises a poloxamer. In some embodiments, the second elution buffer comprises a poloxamer. In some embodiments, the first elution buffer and/or the second elution buffer further comprises between about 0.001%-0.1% (weight/volume) poloxamer. In some embodiments, the poloxamer is poloxamer 188.

In some embodiments of any of the preceding aspects, the first elution buffer and/or the second elution buffer further comprises sucrose. In some embodiments, the first elution buffer comprises sucrose. In some embodiments, the second elution buffer comprises sucrose. In some embodiments, the first elution buffer and/or the second elution buffer further comprises between about 0.1%-5% (weight/volume) sucrose.

In some embodiments of any of the preceding aspects, the first elution buffer and/or the second elution buffer have a pH of about 9.0.

In some embodiments of any of the preceding aspects, c) comprises initially contacting the anion exchange column with an eluant comprising 100% of the first elution buffer and 0% of the second elution buffer and then linearly decreasing the amount of the first elution buffer in the eluant to 0% and linearly increasing the amount of the second elution buffer in the eluant to 100%.

In some embodiments of any of the preceding aspects, c) comprises initially contacting the anion exchange column with an eluant comprising 100% of the first elution buffer and 0% of the second elution buffer and then decreasing the amount of the first elution buffer in the eluant stepwise to 0% and increasing the amount of the second elution buffer in the eluant stepwise to 100%.

In some embodiments of any of the preceding aspects, c) comprises initially contacting the anion exchange column with an eluant comprising 100% of the first elution buffer and 0% of the second elution buffer and then (i) decreasing the amount of the first elution buffer in the eluant to between about 70-90% and increasing the amount of the second elution buffer in the eluant to between about 10-30%; (ii) linearly decreasing the amount of the first elution buffer in the eluant to 0% and linearly increasing the amount of the second elution buffer in the eluant to 100. In some embodiments, c) comprises initially contacting the anion exchange column with an eluant comprising 100% of the first elution buffer and 0% of the second elution buffer and then (i) decreasing the amount of the first elution buffer in the eluant to 80% and increasing the amount of the second elution buffer in the eluant to 20%; (ii) linearly decreasing the amount of the first elution buffer in the eluant to 0% and linearly increasing the amount of the second elution buffer in the eluant to 100. In some embodiments, during (ii), the amount of the first elution buffer in the eluant and the amount of the second elution buffer in the eluant are held constant over about 1-20 column volumes (CV), such as 1, 2, 3, 4, 5, 6, 7, 8, 9, 10, 11, 12, 13, 14, 15, 16, 17, 18, 19, or 20 CV. In some embodiments, the amount of the first elution buffer in the eluant is held constant at between about 40-60% (such as about 50%) and the amount of the second elution buffer in the eluant is held constant at between about 40-60% (such as about 50%).

In some embodiments of any of the preceding aspects, the anion exchange column comprises a strong anion exchange resin. In some embodiments, the strong anion exchange resin comprises trialkyl ammonium chloride or hydroxide. In some embodiments, the strong anion exchange resin comprises dialkyl 2-hydroxyethyl ammonium chloride or hydroxide. In some embodiments, the anion exchange column is functionalized with quaternary amines. In some embodiments, the anion exchange column is functionalized with diethylaminoethanol.

In some embodiments of any of the preceding aspects, the method further comprises, prior to a), (i) providing a solution comprising cells and the plurality of rAAV vector particles; (ii) lysing the cells of the solution to produce a lysate; (iii) filtering the lysate to produce a clarified lysate; and (iv) subjecting the clarified lysate to an affinity chromatography step to provide the mixture. In some embodiments, the cells are HEK293 cells. In some embodiments, the lysate is diluted prior to (iii). In some embodiments, the lysate or clarified lysate is concentrated and/or diafiltered prior to (iii) or (iv). In some embodiments, the clarified lysate is diluted prior to (iv). In some embodiments, the mixture is diluted prior to anion exchange chromatography. In some embodiments, the method further comprises, prior to a), contacting the mixture or a precursor thereof with a cation exchange column.

In some embodiments of any of the preceding aspects, the method further comprises, prior to a), contacting the mixture or a precursor thereof with a cation exchange column.

In some embodiments of any of the preceding aspects, the method further comprises, after c), contacting the eluate with a cation exchange column.

In some embodiments of any of the preceding aspects, the method further comprises, after c), concentrating and/or filtering the rAAV vector particles of the eluate.

In some embodiments of any of the preceding aspects, the method further comprises, after c), using the rAAV vector particles of the eluate, or a portion thereof, in the preparation of a gene therapy composition.

In some embodiments of any of the preceding aspects, the rAAV of the rAAV vector particles are of serotype AAV1, AAV2, AAV3, AAV4, AAV5, AAV6, AAV7, AAV8, AAV9, AAV10, AAV11, AAV12, AAV13, AAVrh32.33, AAVrh8, AAVrh10, AAVrh74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, or AAV PHP.B. In some embodiments, the rAAV of the rAAV vector particles are of serotype AAV5. In some embodiments, the rAAV of the rAAV vector particles is single-stranded rAAV. In some embodiments, the rAAV of the rAAV vector particles is self-complementary rAAV. In some embodiments, the rAAV is a pseudotyped AAV, comprising the AAV capsid protein of one serotype and the AAV ITRs derived from a different serotype. In some embodiments, the rAAV comprises a chimeric AAV capsid, or a humanized AAV capsid.

In some embodiments of any of the preceding aspects, the therapeutic nucleic acid molecule comprises between about 1000-5000 base pairs. In some embodiments, the therapeutic nucleic acid molecule comprises between about 3000-5000 base pairs.

In some embodiments of any of the preceding aspects, the therapeutic nucleic acid molecule comprises a sequence encoding a 21-hydroxylase (210H) protein. In some embodiments, the sequence is a codon-optimized sequence.

In some embodiments of any of the preceding aspects, the therapeutic nucleic acid molecule is operably linked to a regulatory control sequence. In some embodiments, the regulatory control sequence comprises a human cytomegalovirus (CMV) promoter, a chicken 3-actin (CBA) promoter, a Rous sarcoma virus (RSV) LTR promoter/enhancer, an SV40 promoter, a dihydrofolate reductase promoter, a phosphoglycerol kinase promoter, a CMV immediate/early gene enhancer/CBA promoter, a synapsin promoter, CMV-IE promoter/enhancer, a glial fibrillary acidic protein (GFAP) promoter, or a combination thereof. In some embodiments, the regulatory control sequence comprises a CMV immediate/early gene enhancer/CBA promoter and a woodchuck hepatitis virus posttranscriptional regulatory element (WPRE). In some embodiments, the regulatory control sequence comprises a beta-glucuronidase (GUSB) promoter. In some embodiments, the rAAV vector particles disclosed herein comprise additional expression control elements which are operably linked to the transgene. Expression control elements include, for example, appropriate transcription initiation, termination, and enhancer sequences; efficient RNA processing signals such as splicing and polyadenylation (polyA) signals; sequences that stabilize cytoplasmic mRNA; sequences that enhance translation efficiency; sequences that enhance protein stability; and sequences that enhance secretion of the encoded product. In some embodiments, the rAAV vector particles comprise an intron. In some embodiments, the rAAV vector particles comprise a post-transcriptional element, such as a woodchuck hepatitis virus post-transcriptional element (WPRE), a hepatitis B virus posttranscriptional regulatory element (HBVPRE), or a RNA transport element (RTE). In some embodiments, the rAAV vector particles comprise 5′ non-transcribed and 5′ non-translated sequences involved with the initiation of transcription and translation respectively, such as a TATA box, capping sequence, CAAT sequence, enhancer elements, and the like. In some embodiments, the rAAV vector particles comprise an enhancer sequence or upstream activator sequence. In some embodiments, the rAAV vector particles comprise 5′ leader or signal sequences. In some embodiments, the rAAV vector particles comprise a tissue-specific regulatory sequence.

In some embodiments of any of the preceding aspects, the therapeutic nucleic acid molecule is operably linked to a promoter. In some embodiments, the promoter is a cytomegalovirus/0-actin hybrid promoter, PGK promoter, or a promoter specific for expression in an adrenal cortex cell. In some embodiments, the cytomegalovirus/0-actin hybrid promoter is a CAG, CB6, or CBA promoter. In some embodiments, the promoter is a constitutive promoter. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the 3-actin promoter, the phosphoglycerol kinase (PGK) promoter, and the EFla promoter. In some embodiments, the promoter is an inducible promoter. Non-limiting examples of inducible promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-repressible system, the tetracycline-inducible system, the RU486—inducible system and the rapamycin-inducible system. Other types of inducible promoters include those that are regulated by a specific physiological state, e.g., temperature, acute phase, a particular differentiation state of the cell, or a specific cell cycle phase.

In some embodiments of any of the preceding aspects, the therapeutic nucleic acid molecule comprises at least one AAV inverted terminal repeat (ITR). In some embodiments, the at least one ITR is of serotype AAV2.

In some embodiments of any of the preceding aspects, the therapeutic nucleic acid molecule comprises a consensus sequence such as a Kozak sequence.

In some embodiments of any of the preceding aspects, the therapeutic nucleic acid molecule comprises a microRNA (miRNA) binding site, such as an miR-122 binding site.

In some embodiments of any of the preceding aspects, the therapeutic nucleic acid molecule comprises a polyadenylation (polyA) sequence.

In some embodiments of any of the preceding aspects, a method of producing an rAAV comprises contacting a host cell with the therapeutic nucleic acid molecule, or a nucleic acid molecule comprising a sequence encoding the therapeutic nucleic acid molecule or a nucleic acid molecule comprising a sequence complementary or reverse complementary to a sequence encoding the therapeutic nucleic acid molecule, or a plasmids comprising the same.

In some embodiments of any of the preceding aspects, preparation of rAAV particles involves culturing a host cell that contains a nucleic acid sequence encoding an AAV capsid protein or fragment thereof; a functional rep gene; a recombinant AAV vector composed of AAV inverted terminal repeats (ITRs) and an AAV expression cassette comprising the therapeutic nucleic acid molecule, or a sequence encoding the same; and sufficient helper functions to permit packaging of the recombinant AAV vector into the AAV capsid proteins. In some embodiments, the components to be cultured in the host cell to package an rAAV vector in an AAV capsid are provided to the host cell in trans. In some embodiments, any one or more of the required components (e.g., recombinant AAV vector, rep sequences, cap sequences, and/or helper functions) are provided by a stable host cell that has been engineered to contain one or more of the required components. In some embodiments, the host cells are HEK293 cells.

In some embodiments of any of the preceding aspects, a stable host cell will contain the required component(s) under the control of an inducible promoter or a constitutive promoter. In some embodiments, a selected stable host cell contains selected component(s) under the control of a constitutive promoter and other selected component(s) under the control of one or more inducible promoters. For example, a stable host cell may be generated which is derived from 293 cells (which contain El helper functions under the control of a constitutive promoter), but which contain the rep and/or cap proteins under the control of inducible promoters. The recombinant AAV vector, rep sequences, cap sequences, and helper functions required for producing the rAAVs disclosed herein may be delivered to the packaging host cell using any appropriate genetic element (for example, a vector). Further details on methods of preparing rAAV particles are provided in Sambrook et al., Molecular Cloning: A Laboratory Manual, Cold Spring Harbor Press, Cold Spring Harbor, N.Y.; K. Fisher et al, J. Virol., 70:520-532 (1993) and U.S. Pat. No. 5,478,745, the contents of each of which are herein incorporated in its entirety for all purposes.

In some embodiments of any of the preceding aspects, recombinant AAVs are produced using the triple transfection method, as described in U.S. Pat. No. 6,001,650, the contents of which are herein incorporated in its entirety for all purposes. In some embodiments, the recombinant AAVs are produced by transfecting a host cell with a recombinant AAV vector (comprising an AAV expression cassette comprising a therapeutic nucleic acid molecule) to be packaged into AAV particles, an AAV helper function vector, and an accessory function vector.

An AAV helper function vector encodes the “AAV helper function” sequences (i.e., rep and cap), which function in trans for productive AAV replication and encapsidation. Non-limiting examples of AAV helper function vectors include pHLP19 and pRep6cap6 vector, described in U.S. Pat. Nos. 6,001,650 and 6,156,303, respectively, the contents of each of which are herein incorporated in its entirety for all purposes. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some embodiments of any of the preceding aspects, recombinant AAVs are produced using baculovirus vectors. Baculovirus vectors are used to produce recombinant AAVs in insect cells (e.g., Spodoptera frugiperda (Sf9) cells).

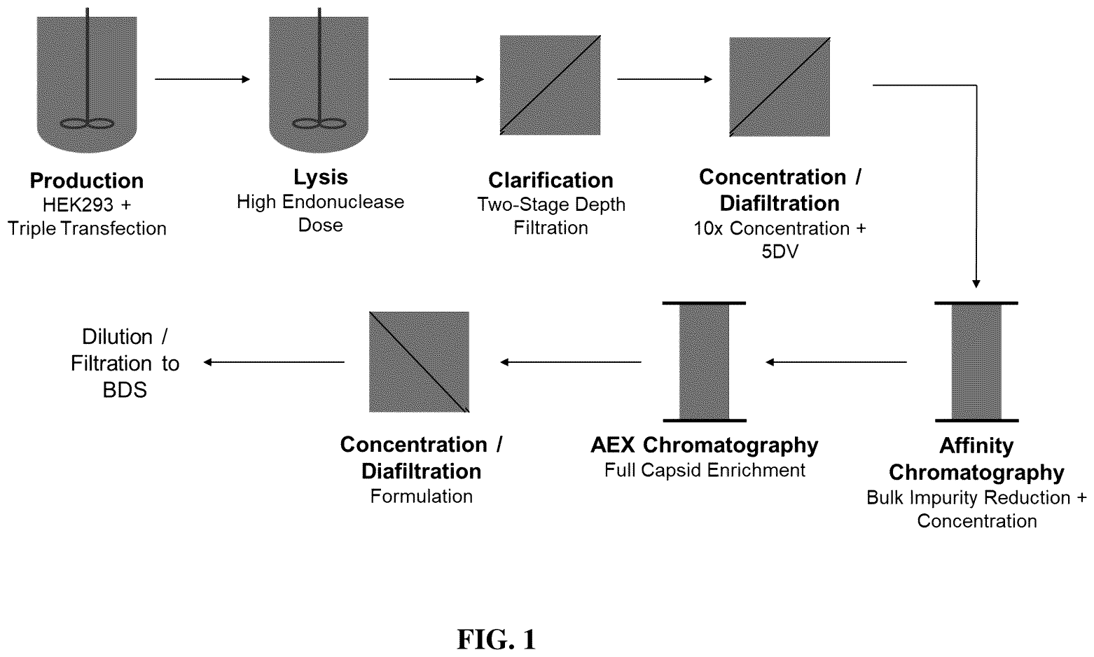

In some embodiments of any of the preceding aspects, following initial production of rAAVs in host cells, the host cells are lysed to provide a lysate. The resultant lysate includes components of the lysed cells as well as rAAV vector particles including the therapeutic nucleic acid molecule of interest, rAAV vector particles not including the therapeutic nucleic acid molecule of interest, and aggregates of various rAAV vector particles. The lysate can optionally be diluted, concentrated, or filtered prior to subsequent processing. The lysate can then be subjected to a clarification process involving single or multi-stage depth filtration, such as two-stage depth filtration, and/or a concentration/diafiltration process to enhance the concentration of the rAAV vector particles of interest. The clarified lysate can then be subjected to a concentration and diafiltration process involving tangential flow filtration. Affinity chromatography can subsequently be performed to remove certain impurities and further concentrate the rAAV vector particles of interest. Anion exchange chromatography can further enhance capsid enrichment, and subsequent additional processing comprising, e.g., cation exchange chromatography, and/or additional concentration/diafiltration, dilution, and filtration can produce a concentrate of rAAV vector particles comprising the therapeutic nucleic acid molecule of interest for subsequent formulation. FIG. 1 shows an overview of an rAAV vector particle production process.

### EXAMPLES

### Example 1: Anion Exchange Chromatography Enrichment of AAV5 Full Capsids with Dual Salt Step, Linear, and Hybrid Elution Gradients

AAV5 vector particles were produced according to scheme shown in FIG. 1. This example describes details of the anion exchange chromatography (AEX) processing step.

AAV5 was prepared using a HEK293 suspension culture that was triple transfected to produce the product of interest, then chemically lysed and clarified by depth filtration and 0.22 micrometer (m) polishing filtration. Clarified lysate was concentrated 10-fold by tangential flow filtration, then purified by affinity chromatography (FIG. 1). For anion exchange chromatography, anion exchange (AEX) columns were prepared by packing Thermo Poros 50HQ resin to a 20 cm bed height. Affinity eluate was neutralized, then diluted 1:9 in AEX equilibration buffer (20 mM Bis-Tris propane, 0.01% (w/v) poloxamer 188, 1% (w/v) sucrose, pH 9.0 unless otherwise specified) and titrated to pH 9.0+/−0.1. The load material was then loaded at a 4 minute residence time across the prepared and equilibrated AEX columns using a Cytiva AKTA Avant 150 of Cytiva AKTA Pure 150. The columns were then washed with 5 CV equilibration buffer prior to elution. Generally, chromatographic analysis began with 100% Buffer A and 0% Buffer B and proceeded with linear gradient (optionally with a hold of one or more column volumes, CV), step gradient, or a hybrid linear+step gradient method to 0% Buffer A and 100% Buffer B.

Where vector genomes (vg) were determined, vector genome titers were determined based on quantification of the gene of interest by digital droplet PCR (ddPCR). Capsid concentration was determined using a Progen AAV5 Xpress ELISA or Progen AAV5 Titration ELISA. Product aggregation was monitored by relative product diameter and polydispersity using a Wyatt DynaPro Plate Reader.

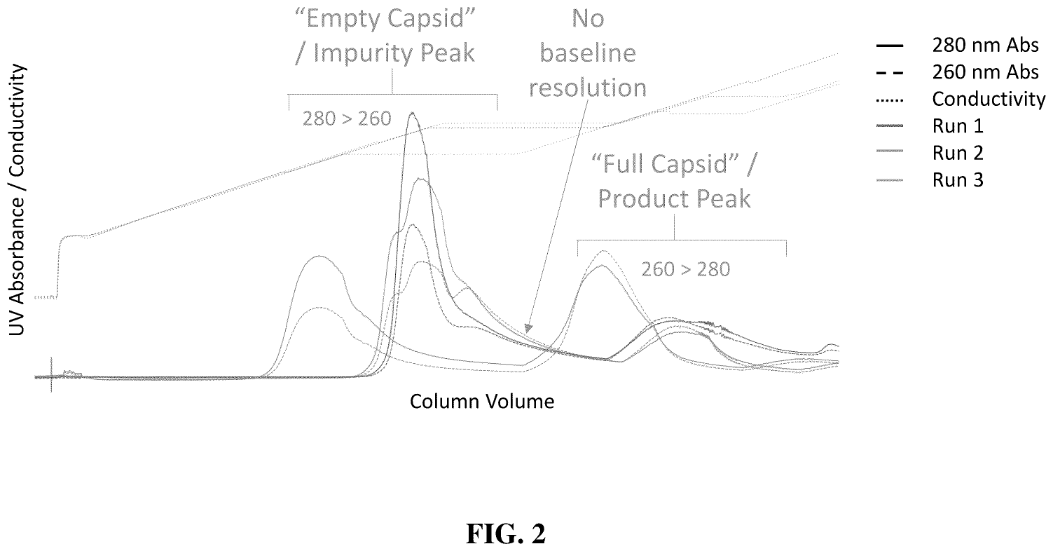

FIG. 2 shows an initial AAV5 AEX chromatogram produced using a linear sodium chloride gradient across a monolithic quaternary amine chromatography column and showing a ˜3× enrichment factor. The capsid has a low vector genome (vg) yield range (˜47%±16%), poor robustness/reproducibility in chromatographic appearance and yield, and a lack of flexibility in available column sizing/required column cycling. The chromatogram includes a peak corresponding to “empty capsids” (e.g., rAAV vector particles that do not include the therapeutic nucleic acid molecule of interest) as well as a peak corresponding to “full capsids” (e.g., rAAV vector particles that include the therapeutic nucleic acid molecule of interest). Improvement of the separation of the full and empty capsids using the AEX process was desirable. Desired features include vg yield of ≥60%, full capsid enrichment factor of ≥3×, minimization of post-AEX aggregation, and no change to vector transduced protein expression.

### Example 2: Single Salt Linear Gradient Screening

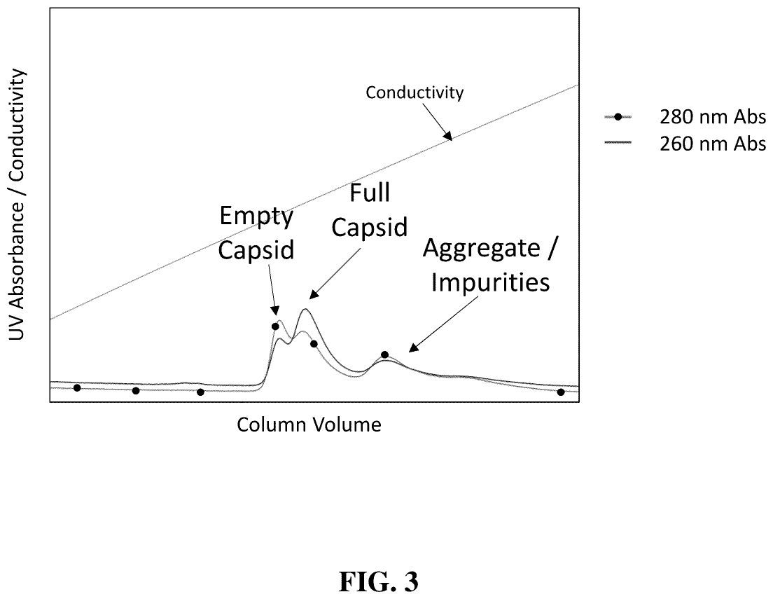

In order to optimize the AEX process, a single salt linear gradient screen was performed. The initial approach involved a transition from quaternary amine (QA) monolith to QA beaded resin in a packed bed to address variability observed with the monolith. To establish the performance of an NaCl linear gradient on the QA packed bed, a 0.66 centimeter (cm) ID×20 cm BH packed bed column was used. The buffer system included 20 mM bis-tris propane, 0.01% weight/volume (w/v) poloxamer 188, and 1% (w/v) sucrose at pH 9.0. FIG. 3 shows the corresponding chromatogram. As shown in FIG. 3, the empty and full capsid peaks are poorly resolved, and a large and poorly resolved impurity peak is also observed.

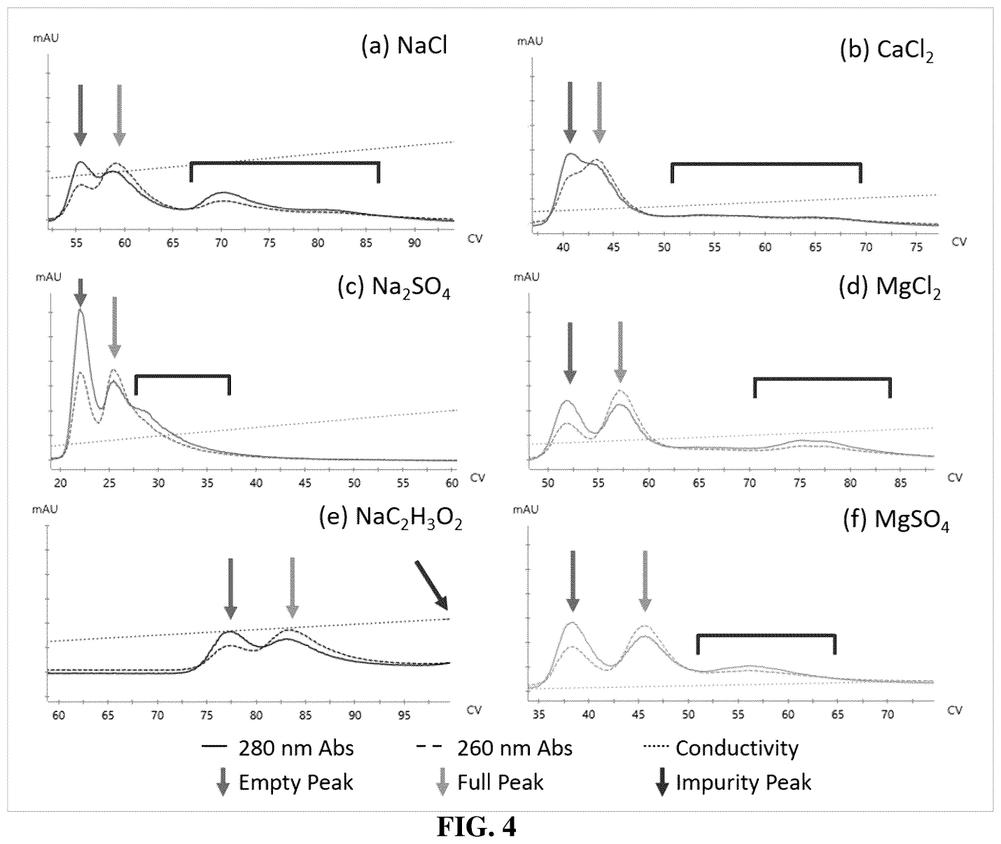

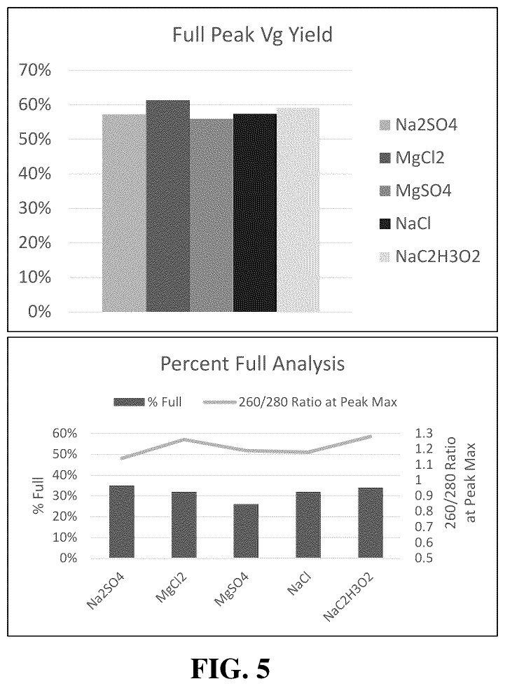

Individual and combinations of monovalent and divalent salt gradients were screened to identify optimal gradient conditions for AAV5 full capsid enrichment. Individual salts screened for comparison to sodium chloride included sodium sulfate, magnesium sulfate, magnesium chloride, calcium chloride, and sodium acetate. Salts were selected based on USP component variability, components available with good safety profile, and to provide a variety in monovalency/divalency and Hofmeister series. FIG. 4 shows chromatograms for the single salt linear gradient screening. Each condition utilized a linear salt gradient for elution, where the salt in Buffer B was varied. Elution conditions were performed in the equilibration buffer system described herein, with the gradient conditions as follows: Panel (a) 0-200 mM sodium chloride, (b) 0-50 mM calcium chloride, (c) 0-120 mM sodium sulfate, (d) 0-50 mM magnesium chloride, (e) 0-200 mM sodium acetate, and (f) 0-37.5 mM magnesium sulfate, each over 100 CV. As shown in FIG. 4, sodium sulfate, magnesium chloride, and magnesium sulfate showed improved empty/full peak resolution, while magnesium chloride and magnesium sulfate showed improved full/impurity peak resolution. Generally, improved resolution of empty and full peaks is apparent using magnesium salts and sodium sulfate. Substantial changes in peak area of impurity peak were observed as a function of the salt gradient used, with no improvement being observed with calcium chloride. Sodium chloride resulted in poorly resolved full and empty peaks, and large peak post-full capsid elution (associated with aggregate). Appearance and intensity of the third peak (impurity peak associated with aggregate) varied as a function of salt gradient used. Minor differences in vg yield and percent full were observed. FIG. 5 shows an analysis of the single salt screening. No major differences in % yield were observed, though the highest yield was observed for magnesium chloride. As the results are based on fractionated and pooled post-run materials, the yields may not be representative. The percent full (ddPCR/Capsid) fell in range of historical processes for all conditions. The ratio of absorbance at 260 nanometers (nm) to absorbance at 280 nm (260/280) at the peak maxima was highest for magnesium chloride and sodium acetate. Aggregate monitoring by dynamic light scattering (DLS) indicated increased capsid aggregation for monovalent anions (chloride and acetate).

### Example 3: Dual Salt Linear Gradient Screening

A dual salt linear gradient screen was performed to determine whether combining multiple salts offers further advantages in resolution, full capsid enrichment, or aggregation. Salt screen conditions were selected to establish identical ionic strength gradients across each condition (0-200 mM ionic strength over 100 CV). Ionic strength was split evenly (e.g., 100 mM of salt 1 and 100 mM of salt 2).

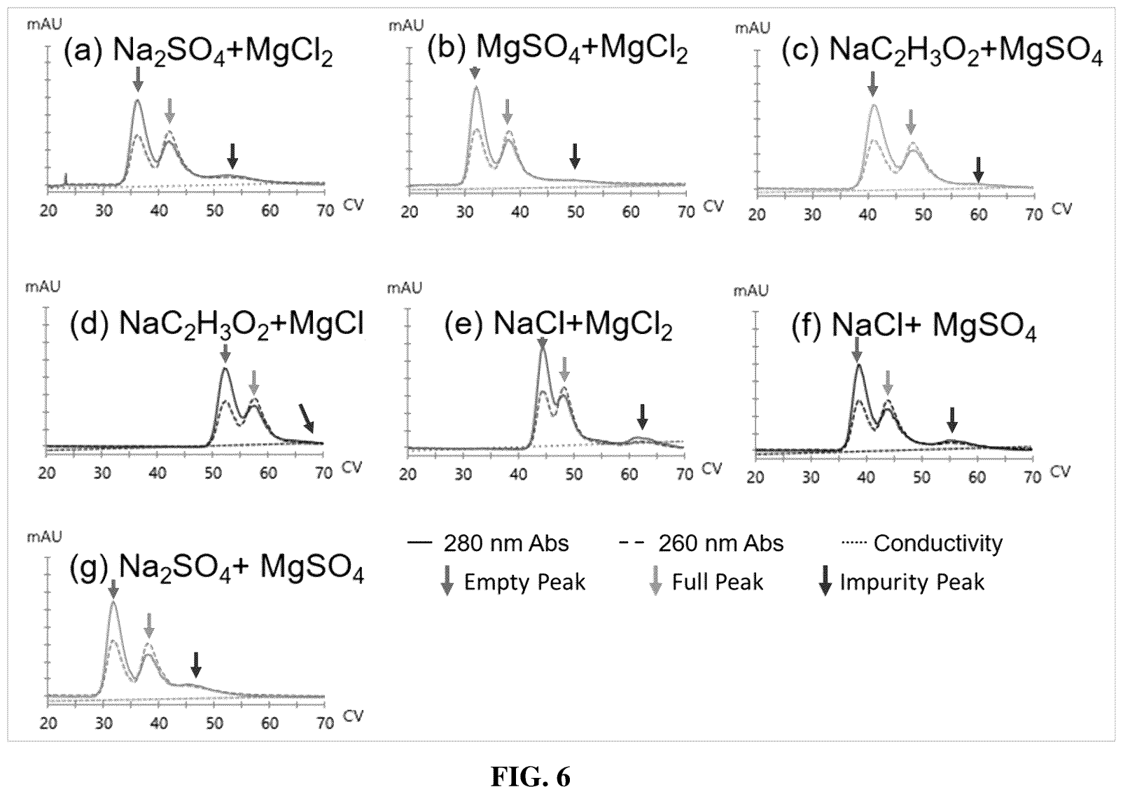

FIG. 6 shows chromatograms for the dual salt linear gradient screening. Results are summarized in Table 1. Each condition utilized a linear salt gradient for elution, where the salts in buffer B was varied. Elution conditions were perform in the equilibration buffer system described above, with the gradient conditions as follows: Panel (a) 0-33.3 mM sodium sulfate+0-33.3 mM magnesium chloride, (b) 0-25 mM magnesium sulfate+0-33.3 mM magnesium chloride, (c) 0-100 mM sodium acetate+0-25 mM magnesium sulfate, (d) 0-100 mM sodium acetate+0-33.3 mM magnesium chloride, (e) 0-100 mM sodium chloride+0-33.3 mM magnesium chloride, (f) 0-100 mM sodium chloride+0-25 mM magnesium sulfate, and (g) 0-33.3 mM sodium sulfate+0-25 mM magnesium sulfate, each over 100 CV. As shown in FIG. 6 and described in Table 1, the presence of sulfate improved empty/full peak resolution. Magnesium sulfate appeared to increase peak area for the empty capsid peak, and decrease impurity peak area. The presence of chloride improved the full/impurity peak resolution. Based on these results, the conditions were narrowed to dual salt conditions (magnesium sulfate+magnesium chloride, sodium acetate+magnesium sulfate, and sodium acetate+magnesium chloride) based on the maximized peak resolution, higher vg yield (>60%), high enrichment factor (>2.5×), and low aggregation (DLS polydispersity 12.5%).

TABLE 1
Dual salt linear gradient screening results

| Salt 1 | Salt 2 | |
|---|---|---|
| Sodium | Magnesium | Improved empty/full |
| sulfate | chloride | peak resolution |
| | | Improved full/impurity |
| | | peak resolution |
| Magnesium | Magnesium | Improved empty/full |
| sulfate | chloride | peak resolution |
| | | Increased peak area for empty capsid |
| | | peak, and decreased impurity peak area |
| | | Improved full/impurity |
| | | peak resolution |
| Sodium | Magnesium | Improved empty/full |
| acetate | sulfate | peak resolution |
| | | Increased peak area for empty capsid |
| | | peak, and decreased impurity peak area |
| Sodium | Magnesium | Improved full/impurity |
| acetate | chloride | peak resolution |
| Sodium | Magnesium | Improved full/impurity |
| chloride | chloride | peak resolution |
| Sodium | Magnesium | Improved empty/full |
| chloride | sulfate | peak resolution |
| | | Increased peak area for empty capsid |
| | | peak, and decreased impurity peak area |
| Sodium | Magnesium | Improved empty/full |
| sulfate | sulfate | peak resolution |
| | | Increased peak area for empty capsid |
| | | peak, and decreased impurity peak area |

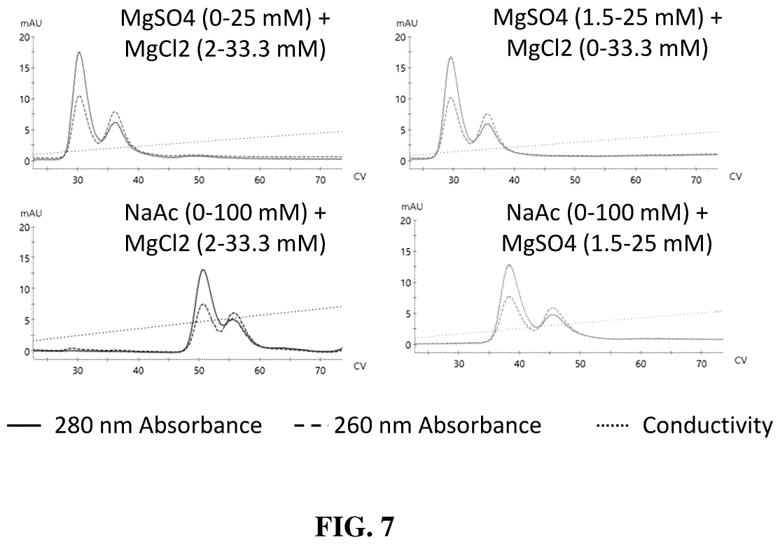

FIG. 7 shows chromatograms from a second dual salt linear gradient screening. A low concentration of a magnesium salt was included in Buffer A was used to improve resolution. The salt combinations were screened for resolution, yield, and aggregation with low ionic strength (6 mM) magnesium salt included in Buffer A. The 280 nm absorbance peak area for the impurity peak was reduced for all conditions screened. The highest vg yields were observed for magnesium chloride and magnesium sulfate gradients. When magnesium chloride was included in Buffer A, the yield was 64%; when magnesium sulfate was included in Buffer A, the yield was 56%. No impurity peak was detected for the magnesium chloride and magnesium sulfate gradient with 1.5 mM concentration of magnesium sulfate in Buffer A. A low aggregate in the full peak was detected by DLS.

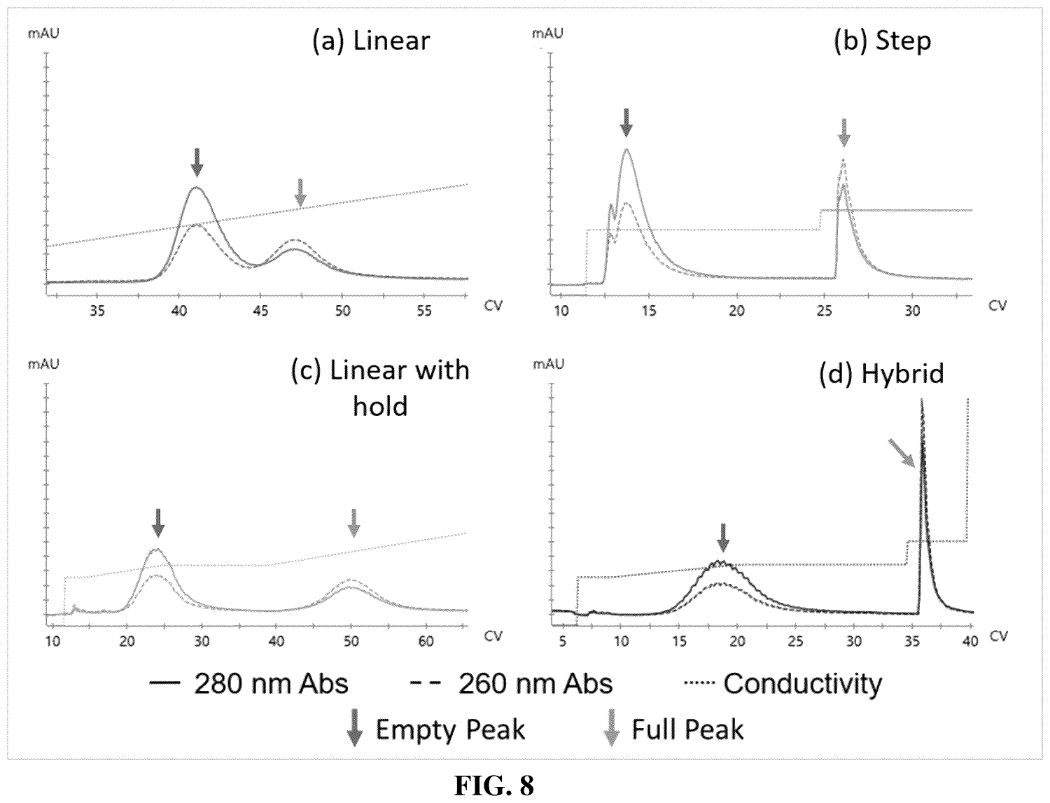

Step, linear, and hybrid elution gradients were subsequently compared. FIG. 8 summarizes the comparison of linear, step, linear with hold, and hybrid elution gradients. Panel (a) used 1.5 mM magnesium sulfate in Buffer A (0% Buffer B) to 25 mM magnesium chloride and 33.3 mM magnesium sulfate in Buffer B (100% Buffer B). A linear gradient was implemented from 0-100% B over 100 CV at a 4 minute residence time. For panels (b)-(d), the following buffer conditions were used: 2 mM magnesium sulfate in Buffer A (0% Buffer B) to 25 mM magnesium chloride, 35 mM magnesium sulfate in Buffer B (100% Buffer B) in 20 mM bis-tris propane, 0.01% (w/v) poloxamer 188, and 1% (w/v) sucrose at pH 9.0. Panel (b) used a step elution strategy, with peak 1/empty capsids eluted at 27% Buffer B for 10 CVs and peak 2/full capsids eluted at 35% Buffer B for 6 CVs. Panel (c) used a linear gradient from 20-50% Buffer B over 60 CV, with a ˜10 CV gradient hold implemented on the backside of peak 1/empty capsid peak at 95% of the 280 nm absorbance peak maximum, followed by a continuation of the linear gradient. Panel (d) utilized a linear gradient from 20-50% Buffer B over 60 CV until the backside of peak 1/empty capsid peak at 85% of the 280 nm absorbance peak maximum was detected, followed by a 15 CV gradient hold, then a step increase to 35% Buffer B for 7 CVs.

For the linear gradient with a hold, 92% vg yield was observed (N=2), with 47% full/4.3× enrichment factor. For the step elution, 83% vg yield was observed with 35% full/3.5× enrichment factor. The evaluation intentionally used varied Buffer B preparation, which showed yield and % full not robust enough for manufacturing. Step elution decreased polydispersity in the product pool substantially (20.6% vs. 11.4%). Hybrid elution showed 94.8% yield with 49% full/4.9× enrichment factor and 11.9% polydispersity. Table 2 summarizes these results.

TABLE 2
Comparison of step, linear, and hybrid elution gradients

| | | | Vg | DLS | |
|---|---|---|---|---|---|
| | | Variability | yield | polydispersity | Enrichment |
| Gradient mode | N | risk | (%) | (%) | factor |
| Linear | 1 | Low | 54% | 17.0% | 3.5X |
| Linear with hold | 2 | Low | 92% | 20.6% | 4.3X |
| Step | 1 | High | 83% | 11.4% | 3.5X |
| Hybrid | 1 | Low | 95% | 11.9% | 4.9X |

Overall, the optimization of gradient salts coupled with the transition from an AEX monolith to a packed bed AEX column substantially improved empty/full resolution. Implementing an optimized hybrid gradient resulted in vector yield increase from 47% to >90%, 4-5× full capsid enrichment factor, and low aggregation based on DLS polydispersity. The results indicate that some aggregate forms during the AEX process, and can be mitigated via addition of magnesium salts and/or transitioning to a step or hybrid gradient.

### Example 4

AAV5 was produced using a HEK293 suspension culture that was triple transfected, lysed, and harvested by depth filtration and 0.22 μm polishing filtration. Two upstream conditions to produce the same AAV5 vector were used to generate starting material for this work: feed stream 1 (FS1) and feed stream 2 (FS2). The clarified lysate was then concentrated 10-fold and diafiltered by tangential flow filtration. Bulk impurity reduction and concentration were subsequently performed by affinity chromatography and the neutralized eluate was forward processed to AEX chromatography. Columns with 0.66 cm inner diameter (ID)×20 cm bed height (BH) packed with Thermo Poros 50HQ resin were used for the initial single salt and dual salt screening conditions, with all steps performed at a 6-minute residence time (200 cm/hr) in downflow. All screening runs were performed using FS1, where percent full was estimated at ˜20% full by vector genome titer determined by ddPCR divided total capsid titer determined by Capsid ELISA. The linear velocity was selected to support pressure drop profiles to enable scale-up. All screening runs were performed at a capsid (cp) load factor of ˜−2×1013 cp/mL column volume (CV), determined by capsid ELISA.

The buffer composition for both the equilibration (Buffer A) and elution gradient (Buffer B) buffers for the AEX runs included 20 mM bis-tris propane, 1% (w/v) sucrose, 0.01% (w/v) poloxamer 188, pH 9.0, with additional salts in each dictated by condition indicated in Tables 3 and 4. For example, for the first condition described in Table 4, the composition for Buffer A is 20 mM bis-tris propane, 2 mM magnesium chloride, 1% (w/v) sucrose, 0.01% (w/v) poloxamer 188, pH 9.0. For Buffer B, the composition is 20 mM bis-tris propane, 33.3 mM magnesium chloride, 25 mM magnesium sulfate, 1% (w/v) sucrose, 0.01% (w/v) poloxamer 188, pH 9.0. For each AEX chromatography run, neutralized affinity eluate was diluted 10-fold with AEX equilibration buffer as dictated by each experimental condition, then titrated with 1M bis-tris propane to pH 9.0±0.1. The AEX column was equilibrated with 5-10 CV Buffer A, followed by the prepared load, then subsequently washed with 5-10 CV Buffer A. For all linear gradient conditions, linear gradients were performed over 100 CV for screening purposes. Fixed volume fractions were collected throughout the elution gradient, and neutralized fractions were pooled based on the chromatogram to represent 3 regions of the elution: Peak 1 (P1, “empty capsid peak”), Peak 2 (P2, “full capsid peak”), and Peak 3 (P3, “impurity peak”). Each pool was further analyzed for aggregation by dynamic light scattering (DLS).

For the initial single salt screening, six gradient salts (sodium chloride [NaCl], sodium sulfate [Na2SO4], sodium acetate [NaC2H3O2], magnesium chloride [MgCl2], magnesium sulfate [MgSO4], and calcium chloride [CaCl2)]) were screened by linear gradient elution as described in Table 3. Salts selected for the initial gradient screening were selected to allow for comparisons based on valency and chaotropic/kosmotropic behavior for both anion and cations in addition to limits based on toxicity, cost, and availability of multi-compendial grade materials, aspects that become important for full-scale manufacturing in a current Good Manufacturing Practices (cGMP) environment.

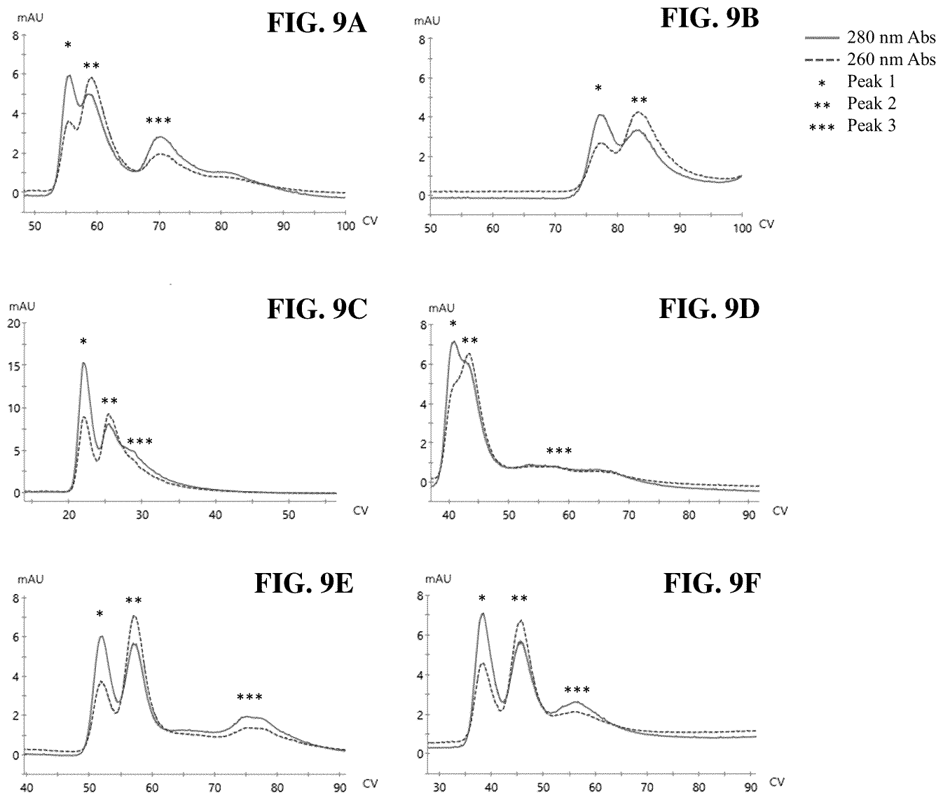

Chromatograms for the initial single salt screening are reported in FIGS. 9A-9F. While the intent of this study included maintaining a consistent ionic strength gradient slope, the initial run (Na2SO4) was performed at a steeper slope (3.6 mM/CV). The elution profile indicated that a smaller ionic strength range could safely be used and still allow for full elution of all product peaks, and thus was subsequently reduced to 1.5-2.0 mM/CV for all remaining runs. Conditions for the initial screening were compared and narrowed primarily by comparison of the calculated resolution, 280 nm absorbance (A280) peak area for the three distinct peaks, and the ratio of the 260 nm absorbance/280 nm absorbance at the 260 nm absorbance peak max for P2 (A260/A280), a measure that correlates to relative % full capsids from run to run, as summarized in Table 3. Hydrodynamic radius and polydispersity by DLS results are also reported in Table 3. Hydrodynamic radius was collected as a means of confirming that the majority of the particles in each pool aligned with the expected diameter of ˜25 nm without additional larger diameter peaks (indicating larger distinct aggregate species), while the polydispersity was collected to indicate the degree of homogeneity in the average diameter reported, where <15% polydispersity indicates a high likelihood of homogeneity (i.e. primarily monomer) and >30% indicates a high likelihood of heterogeneity (i.e. additional species other than the monomer likely contribute to the diameter).

As a result of the very low P1/P2 (P1/P2) resolution observed using a CaCl2) gradient (0.4, compared to 0.5 for NaCl and NaC2H3O2, and >0.7 for all other conditions), CaCl2) was removed from experimental conditions after the single salt gradient screening. Additionally, high P1/P2 resolution observed for the magnesium salt gradients (0.8 for MgCl2 and 0.9 for MgSO4) resulted in limiting further evaluations to only conditions that included at least one magnesium salt.

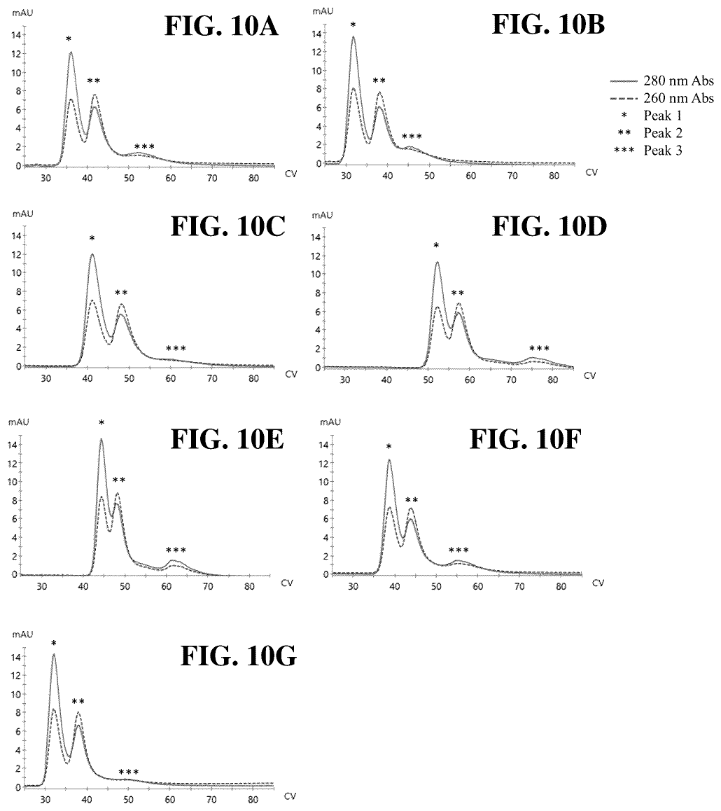

For dual salt elution screening, the AEX chromatography was run similarly to what is outlined above, with modified Buffer B as dictated by condition (Table 3). Here, dual salt screening runs utilized a Buffer B at a target ionic strength of 200 mM split evenly between the two salts (100 mM salt 1, 100 mM salt 2) with a fixed linear gradient slope based on ionic strength of 2.0 mM/CV. Fixing the gradient slope based on ionic strength (instead of the traditional salt concentration) is important for comparative analyses when dealing with various salts of different valencies. Chromatograms for the dual salt screening conditions are shown in FIGS. 10A-10G. The resulting calculated A280 peak areas, resolutions, A260/A280 ratios, and DLS results for each condition are reported in Table 3. Based primarily on P1/P2 resolution, four dual salt conditions were identified as lead candidates: MgCl2+MgSO4, Na2SO4+MgCl2, NaC2H3O2+MgCl2, and NaC2H3O2+MgSO4.

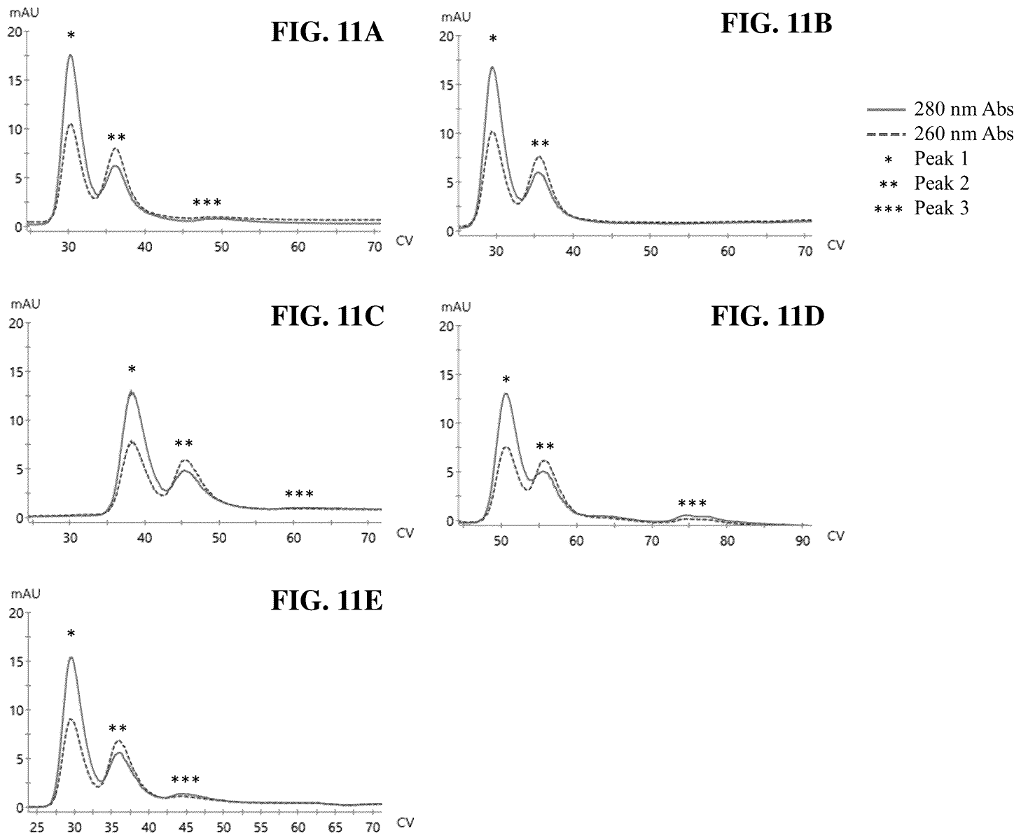

Without being bound by a theory, it is thought that the incorporation of low ionic strength MgCl2 either in Buffer A or at a constant concentration in Buffers A and B improves resolution. We evaluated the impact of a low concentration of both MgCl2 and MgSO4 in the load and during equilibration by performing additional screening runs with either 1.5 mM MgSO4 or 2 mM MgCl2 (6 mM ionic strength for each condition) in Buffer A. Both MgCl2 and MgSO4 in the load dilution/equilibration buffer were tested for the MgCl2+MgSO4 dual gradient, only MgCl2 was tested for Na2SO4+MgCl2 and NaC2H3O2+MgCl2, and only MgSO4 for NaC2H3O2+MgSO4. The gradient salts, resulting calculated A280 peak areas, resolutions, A260/A280 ratios, and DLS results for each condition are reported in Table 4. Chromatograms for the third round of screening are reported in FIGS. 11A-11E. Based on the resolution, P2 aggregation as measured by DLS polydispersity, and no observed P3 (unique among the conditions tested, as seen in the chromatograms in FIGS. 9A-9F, 10A-10G, and 11A-11E), MgSO4+MgCl2 with low concentration MgSO4 in Buffer A was selected as the lead condition for further development. A DLS polydispersity of >30% is generally indicative of a polydisperse population, and as a result the 17% polydispersity condition observed for this run did not indicate significant aggregation in this pool.

While the optimized salt gradient selected achieved substantial improvement in resolution between the full and empty capsid species and resulted in no observable P3, baseline resolution of peaks 1 and 2 (primarily empty and full capsids, respectively) was not achieved with the use of a linear gradient alone. To further approach baseline resolution, the method was modified with a shallower salt gradient from 2 mM ionic strength per CV to 1 mM ionic strength per CV and to include a 15 CV gradient hold on the backside of P1 that is initiated based on 280 nm absorbance. For this work, the column was equilibrated, loaded, and washed as above. A second wash at 80% Buffer A, 20% Buffer B for 3 CV was implemented to shorten the elution gradient in the region where no product elution was observed.

To allow full baseline separation between P1 and P2, a gradient hold was initiated on the backside of P1 for 15 CV, where two hold triggers were compared (85% or 95% of the 280 nm absorbance peak max). While gradient mode development is not in the scope of the work presented here, a linear gradient through the elution of P1 was implemented due to high variability in separation and reduced yield and final percent full when isocratic elution of P1 was used. Additionally, P2 was eluted using a step at 35% B to increase the concentration of the eluate collected, reduce processing time, and improve % full and yield. This gradient was subsequently evaluated at two additional load factors at the 0.66 cm ID column scale (5×1013 cp/mL CV and 1.5×1014 cp/mL CV) to evaluate more feasible, higher load factors for an at-scale process operated with a single cycle of AEX processing.

Aggregation by DLS in addition to vector genome (vg) and cp yields for these runs are reported in Table 5 (Runs A through D). Polydispersity for Run C was notably higher than other runs at ˜40% vs. the typical range of 11-25%. The root cause of this observation is unclear and is thought to be a result of sampling or pool handling during processing or analysis. As slightly higher vg yield was observed at 95% of peak max, these elution conditions were applied at an increased load factor of 4.5×1014 cp/mL with a gradient hold, with the resulting analytical data presented in Table 5 (Run E). In parallel to the above work, the process was scaled up to a 3.5 cm ID column scale and loaded at 2×1013 cp/mL to generate sufficient material to forward process and assess potency. An 85% peak max gradient hold was implemented for this run, as the analysis for Runs A-D was incomplete prior to initiation. Results of the scaled run are presented in Table 5 (Run F). Finally, the process was scaled up to a 5 cm ID×20 cm BH AEX column at 7×1013 cp/mL with the 95% peak max gradient hold, with the resulting analytical data presented in Table 5 (Run G). It should be noted that for this run, Buffer B magnesium salt composition was reduced from 25 mM MgSO4 and 35 mM MgCl2 to 13.5 mM MgSO4 and 17.5 mM MgCl2 with the linear gradient adjusted accordingly to deliver the same gradient conditions during the linear gradient and gradient hold (Wash 2 modified to 40% B for 5 CV, linear gradient adjusted to 40%-100% B over 60 CV, isocratic elution of the full peak adjusted to 70% B).

Robustness of this process was further assessed by forward processing a second feed stream, FS2, where percent full was estimated at ˜10% full by ddPCR/Capsid ELISA. The lower percent full at load increased the size of P1 relative to P2, and as a result the peak max gradient hold was shifted to 75% to accommodate for the change. Additionally, the isocratic elution of the full peak was increased from 70% B to 75% B to tighten the eluate peak. Three runs were performed to demonstrate performance of this step with FS2: one small scale run performed on a 0.66 cm ID×20 cm BH AEX column at 3.9×1014 cp/mL (Run H), and two subsequent scale-up runs performed on a 2.6 cm ID×20 cm BH AEX column at 2.0×1014 cp/mL (Run I) and 3.5×1014 cp/mL (Run J). Results of the three runs performed from FS2 are presented in Table 5.

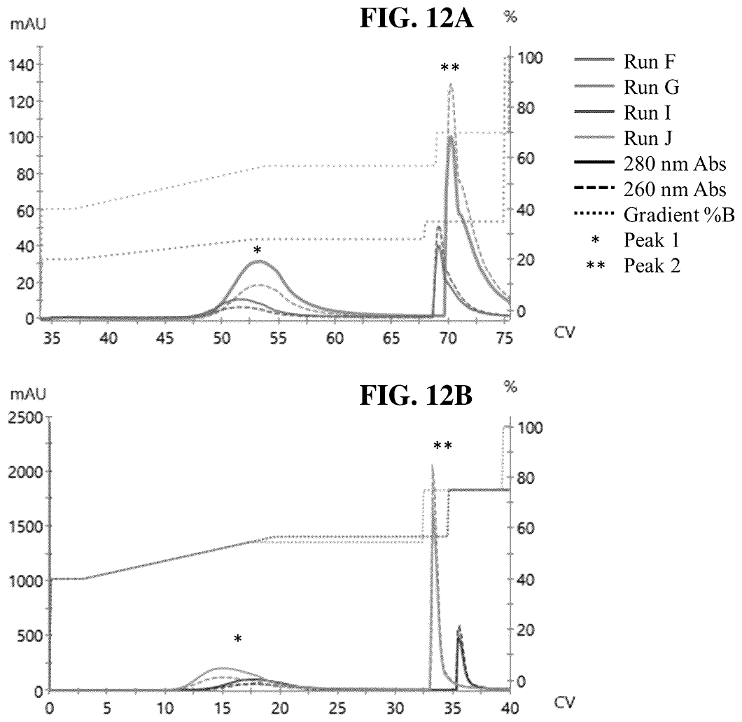

To assess the product quality of the final P2 pool from the revised process, the larger scale runs (Run F, Run G, Run I, and Run J) were concentrated and diafiltered into product formulation buffer, and the resulting product quality was compared for FS1 and FS2 runs. Representative elution chromatograms for the large scale runs are shown in FIGS. 12A-12B. Product quality for the resulting pools was compared using percent full vector by analytical ultracentrifugation (AUC), and aggregation by DLS. Percent full by AUC post-formulation was measured at 75% and 79% full for FS1 Runs F and G, respectively, and 51% and 68% full for FS2 Runs I and J, respectively. With respect to aggregation, hydrodynamic radius measured at 13.6 nm and 13.8 nm, respectively, for FS1 Runs F and G, and 14.0 nm for both FS2 Runs I and J. Polydispersity of the observed peak by DLS for the FS1 runs G and H resulted in 20.62% and 12.07% polydispersity, respectively, and FS2 runs I and J resulted in 24.21% and 11.90% polydispersity, respectively.

Screening of single and dual salt linear gradients for AAV5 resulted in substantial insight into key factors for achieving resolution between full and empty AAV capsids by AEX chromatography. Changes to resolution between peaks 1 and 2 and the formation of P3 as a function of selected salt demonstrated correlations between resolution and chaotropic/kosmotropic nature of each salt, and the relationship between capsid aggregation and resolution. This effect is most clearly demonstrated in the single salt screening assessments, where more kosmotropic ions tended to result in both higher P1/P2 resolution, and lower observed aggregate in P2 and P3 as observed by DLS polydispersity. It is hypothesized that the on-column degradation product observed here is a capsid aggregate species, as supported by the higher polydispersity seen in P3 compared to P1 and P2. Without being bound to a theory, it is thought that these results are promoted by the tendency for aggregates to form at low ionic strength conditions similar to those required for AEX binding. It is further suspected that the aggregate is reversible, given that DLS results suggest that the majority of the product in P3 is monomeric, as supported by work demonstrating a reduction in AAV aggregate upon dilution by DLS, though notably this was demonstrated specifically for AAV2.

Without being bound by a theory, it is thought that the reduced polydispersity in the presence of kosmotropic ions is a result of reduced aggregation during chromatography with increasingly kosmotropic salt gradients, as indicated in Table 3 and demonstrated by observed chromatographs in FIGS. 9A-9F. While the mechanism for this dissociation is not clear, this relationship appeared to be true for both cations and anions. The inclusion of Mg2+ salts showed substantial improvement in resolution over both Na+ and Ca2+ salts, where Mg2+>Ca2+>Na+in the Hofmeister series moving from most to least kosmotropic. The difference in P1/P2 resolution in single salt screenings (0.4 for CaCl2) vs. 0.8 for MgCl2) was particularly striking. Without being bound by a theory, it is thought that divalency alone may not necessarily influence resolution, or this observation may be an effect of the more kosmotropic nature of Mg2+.

Similarly, an assessment of the anions used in the single salt screening suggested that the resolution of the full and empty capsid peaks improved with more kosmotropic anions. When comparing P1/P2 resolution for the sodium salts with varied anions (NaCl at a resolution of 0.5, NaC2H3O2 at 0.5, and Na2SO4 at 0.9), the results again suggest a possible influence based on the kosmotropic nature of the anions, where sulfate>acetate>chloride in the Hofmeister series moving from most to least kosmotropic. This case may support purely a difference based on divalency vs. monovalency. However, taken together with the assessment of the cations, the full dataset suggests improved resolution with more kosmotropic salts. While toxicity, availability of multi-compendial grade versions, and cost of some of the most highly kosmotropic salts may limit implementation for large-scale AAV purification, this assessment can help guide the screening of gradient salts and conditions to identify optimal resolution under these constraints.

The observations from this work lead to the hypothesis that mitigating on-column aggregation, as supported by the reduction in P3 formation and reduced DLS polydispersity observed in the selected high-resolution condition, improves resolution empty vs. full capsid resolution by reducing co-elution of the two species. It is suspected that interactions of the capsids with kosmotropic ions is the likely cause of this observation. Hofmeister series effects have been demonstrated to be caused by interaction between proteins and ions, and the existing best understanding suggests ions that are higher in the Hofmeister series (i.e. moving more towards kosmotropes) are more likely to break “expanded structure”-like water clusters between proteinaceous molecules or otherwise impact the hydration layer at the contact surface of proteins, or in this case, viral capsids. Based on this theory, two possible mechanisms were postulated for how kosmotropic salt ions higher in the Hofmeister series could potentially mitigate aggregation in these low salt, high pH AEX environments. First, highly kosmotropic ions are known to reduce water solvation of protein surfaces by themselves being preferentially hydrated. This phenomenon may allow increased stabilizing interactions between the remaining salt ions in the low ionic strength environment with the capsid surfaces. Second, strong kosmotropes are known to increase the melting temperature of non-hydrophobic proteins by increasing the surface tension of the solution. These factors may offer a potential mechanism for the observed decrease in aggregation in the presence of kosmotropic salts. It is hypothesized that AAV capsids proteins have a natural tendency to partially unfold in high pH/low salt environments, and this partially unfolded (increased exposed surface area) state becomes unfavorable upon the addition of kosmotropes through increased surface tension. The increase in melting temperature may allow an overall increase in stability of the non-hydrophobic regions, lowering the likelihood of unfolding and/or aggregation. Alternatively, the presence of kosmotropes inhibits interaction of capsids in this unfolded state, preventing aggregation.

The results presented here further demonstrate the synergistic effects of implementing a dual salt linear gradient and expands the benefit of this effect through more extensive screening of ion-specific effects. Based on the single salt screening results, all dual salt conditions tested included at least one magnesium salt due to the dramatically improved resolution between peaks 1 and 2. Using this criterion, in addition to the removal of CaCl2) from consideration in the dual salt screenings, the range of P1/P2 resolution increased from 0.4-0.9 in the single salt screening to 0.7-1.2 in the dual salt screening (Table 3). Using the data generated from both the single salt and dual salt screenings, P1/P2 resolutions were compared by analysis of variance (ANOVA) as a function of whether each anion or cation tested was present. From this analysis, gradients that included a magnesium cation and/or a sulfate anion resulted in statistically higher P1/P2 resolution (p=0.0034 and p=0.0031, respectively). The addition of the dual salt screening resolution experiments further supported the single salt screening findings that the inclusion of kosmotropic anions and cations improves P1/P2 resolution.

A similar analysis was performed for P2/P3 resolution. No statistically significant difference was observed as a function of the cation; however, a moderately significant increase was observed when chloride anions were included in the gradient (p=0.0491). The benefit of both sulfate for P1/P2 resolution and chloride for P2/P3 resolution in addition to the high-resolution performance observed for the dual MgSO4/MgCl2 condition compared to its single salt counterparts supports the hypothesis that dual salt gradients benefitted from the additive effects of each individual ion. While no specific trends emerged based on divalency or ion, there were notable differences in the intensity of P3 from condition to condition. A decrease in P3 intensity was notably not correlated with higher polydispersity or hydrodynamic radius by DLS in P2, as might be expected if this observation was a result of poor resolution between P2 and P3. This again suggests that P3 may be an on-column degradation product that may be mitigated by appropriate salt gradient.

Two criteria were used to select lead candidates to maximize resolution and minimize possible on-column degradation (presumed to be aggregation), which was suspected to be causing poor separation of the empty and full species: P1/P2 resolution ≥1.0, P2 DLS polydispersity <12%, resulting in three lead dual salt gradient candidates: MgSO4+MgCl2, MgSO4+NaC2H3O2, and Na2SO4+MgSO4. A fourth condition, MgCl2+NaC2H3O2, was also screened given very high P2/P3 resolution, low P2 DLS polydispersity, and low P3 integrated area observed.

To further optimize resolution between the empty (P1) and full (P2) capsid peaks, the introduction of a low concentration of either 2 mM MgCl2 or 1.5 mM MgSO4 in the load dilution buffer and buffer A, the column equilibration buffer, for the four lead candidates was tested. This study was limited to only magnesium salts already present in the gradient for a total of five conditions (the MgSO4+MgCl2 gradient was tested with both salts individually in Buffer A). With the introduction of a low concentration of magnesium salt in buffer A, the P2 A260/A280 ratio at peak max increased for all conditions (see Table 4) compared to gradients without initial magnesium reported in Table 3.

While P1/P2 resolution did not substantially change with the addition of initial magnesium salt, the inclusion of initial magnesium salt appeared to reduce the intensity of P3 and MgSO4, the more kosmotropic of the two magnesium salts, appeared to further reduce the intensity of P3 to near undetectable levels in two of the three initial MgSO4 conditions tested, and reduced compared to the conditions with no initial magnesium shown in FIGS. 10A-10G. Notably, P3 became undetectable altogether for the MgSO4+MgCl2 gradient with initial MgSO4. P2 DLS polydispersity increased slightly for this condition from 11.6% to 17.0% without and with initial MgSO4, respectively. Despite this increase, the increase in P2 A260/A280 ratio from 1.21 to 1.27 without and with initial MgSO4, respectively, suggests that separation was not negatively impacted, and potentially modestly improved. The inclusion of initial MgCl2 resulted in varied responses that appeared to depend on the second salt in the gradient. In the MgSO4+MgCl2 gradient, an increase in P3 integrated area, P2 and P3 polydispersity, and P2/P3 resolution was observed, suggesting an increase in the formation of the on-column degradation product and simultaneous improvement in its separation from the product peak. Conversely, a decrease in the P2/P3 resolution was observed for the MgCl2+NaC2H3O2 condition, with no substantial change to any other results. The improved P2 A260/A280 ratio and reduced P3 integrated area observed in conditions with initial MgSO4 is hypothesized to result from reduced capsid aggregation. In this condition, MgSO4 acts to stabilize AAV in the low ionic strength buffer matrix after load dilution.

Based on high P1/P2 resolution coupled with a lack of detectable P3, MgSO4+MgCl2 with initial MgSO4 was selected as the final gradient condition. Because baseline resolution was not achieved as a result of the gradient alone as observed in FIGS. 11A-11E, the slope was decreased from 2 mM ionic strength/CV to 1 mM ionic strength/CV, and a gradient hold was implemented as discussed in the results section above. The gradient hold was required for this step to achieve desired separation due to lack of baseline resolution with a linear gradient. This final process was implemented for FS1 (˜20% full) and compared with varied capsid load factor, chromatography scale, and trigger point for the gradient hold on the back side of P1. Implementation of an isocratic elution for P2 resulted in improvements in processing time both during the AEX step via shorter elution of the product peak, and more dramatically during the subsequent concentration step due to a substantial increase in peak concentration post-elution (100-200-fold concentration for the initial process vs. 3-5-fold for the optimized process). Based on observed chromatographic performance, no substantial difference was observed between the two peak hold triggers (85% and 95% of peak max) tested, with an average A260/A280 ratio at peak max of 1.30±0.01 for 85% and 1.29±0.03 for 95%, shown in Table 5. Vector genome yield and percent full comparisons for FS1 runs (Run F and G) further supported equivalency, with yields at 71%±8% and 75%±15% and percent full at 69%±5% and 69%±8% for 85% and 95% holds, respectively. Similarly, no substantial difference was observed as a function of scale. While insufficient replicates were available to establish a statistical correlation, visual comparison indicates that vector genome yield increases with increasing capsid load factor, while calculated percent full based on vector genome titer divided by capsid titer was unaffected. The enrichment factor, defined as the percent full in the eluate pool divided by the percent full in the feed, is tabulated in Table 5. For this feed stream, a calculated enrichment factor of 3.8±0.3 and 3.4±0.2 for the 85% and 95% of peak max conditions, respectively, was achieved, showing substantial enrichment of full capsids across this process step.

The ability of this process test to enrich from a lower percent full at load was subsequently evaluated via purification from FS2 material (˜10% full). Two modifications were made to these runs: (1) the peak 1 hold trigger was reduced from 95% of the 280 nm absorbance Peak 1 peak max to 75% to accommodate the higher empty to full peak ratio with the lower percent full material, and (2) the step to elute the full peak was slightly adjusted from 30% Buffer A and 70% Buffer B (the equivalent of 35% Buffer B with the initial Buffer B composition) to 25% Buffer A and 75% Buffer B to sharpen the peak shape of the eluted full peak. Tabulated results for the three runs performed under these conditions (Runs H, I, and J) are shown in Table 5. The reduced percent full in the feed resulted in an overall reduction in the percent full in the eluate condition, as indicated by the average A260/A280 ratio of 1.20±0.03 and percent full by ddPCR/Capsid ELISA reported concentration of 52%±2%. Though the overall percent full was lower, the enrichment factor observed was calculated at 5.2±0.2, a substantial enrichment factor with comparable yield to the initial feed material tested at an average vector genome yield of 67%±8%.

Finally, the full capsid enrichment step with optimized gradient salts was compared to the initial AEX step used in-house. Runs G, F, I, and J were processed at a scale sufficient to forward process the neutralized P2 pool through concentration and diafiltration to generate formulated product pool. Selected product quality attributes for the four product pools were then compared. The high enrichment factors and achieved percent full using this process step are demonstrated by both the calculated percent full based on vector genome titer divided by capsid particle titer shown in Table 5 and by the average percent full by AUC post-formulation (77% for FS1 runs and 60% for FS2 runs). Comparisons for aggregation were performed by DLS assessment. DLS analysis indicated consistently low aggregate levels in all for runs forward processed, with an average hydrodynamic radius of 13.7 nm for the FS1 runs, and 14.0 nm for the FS2 runs. Polydispersity of the observed peak by DLS additionally did not indicate a highly heterogeneous population (polydispersity <30%) for either feed conditions. Combined, these results suggest that the optimized AEX method developed here enables a process step with high vg yield, enhanced full capsid enrichment, and low aggregation.

In conclusion, a thorough screening of gradient salts for AAV full capsid enrichment by AEX and the implementation of dual salt gradients to enable mixed ion elution resulted in substantial improvements in full vs. empty capsid separation. The results show that implementation of kosmotropic ions improved resolution between empty and full capsids and reduced the formation of the on-column degradation product eluting in P3. Without being bound to a theory, it is thought that on-column degradation product is a reversible aggregate of empty and full capsids that appears to be mitigated with the inclusion of more strongly kosmotropic ions. This applied to both cations, as evident by improvements observed with the inclusion of magnesium salts, and anions, as shown with the improvements observed with the inclusion of sulfate. Finally, it was determined that once formed, P3 can be further resolved from the main product peak with the inclusion of chloride as a gradient salt, supporting the use of both a more kosmotropic anion (i.e., sulfate) and chloride. The final dual salt approach combined the advantages of both minimizing the formation of P3 with kosmotropes and separating any degraded product that did form with chloride, resulting in a final process step that improved vector genome yield, percent full, and aggregate profile for AAV5.

TABLE 3
Single and Dual Salt Screening

| | Ionic Strength | | | P1/P2/P3 A₂₈₀ | Peak 2 | | |
|---|---|---|---|---|---|---|---|
| | Gradient | | | Integrated | A₂₆₀/ | Peak 2 DLS | Peak 3 DLS |
| | Slope | P1/P2 | P2/P3 | Area | A₂₈₀ | radius (nm)/% | radius (nm)/% |
| Gradient Salt | (mM/CV) | Resolution | Resolution | (ml* mAU) | ratio | polydispersity | polydispersity |
| 200 mM NaCl | 2.0 | 0.5 | 1.1 | 120/176/208 | 1.18 | 12.9 nm/13.7% | 16.2 nm/32.4% |
| 200 mM NaC₂H₃O₂ | 2.0 | 0.5 | NA^{a} | 134/188/NA^{a} | 1.28 | 16.2 nm/28.7% | NA^{a} |
| 300 mM Na₂SO₄ | 3.6 | 0.8 | 0.3 | 230/140/188 | 1.14 | 13.2 nm/8.7% | 13.6 nm/19.7% |
| 50 mM CaCl₂ | 1.5 | 0.4 | 0.6 | 153/135/99 | 1.10 | NA^{b} | NA^{b} |
| 50 mM MgCl₂ | 1.5 | 0.8 | 0.9 | 141/180/180 | 1.26 | 12.6 nm/7.2% | 13.7 nm/31.0% |
| 100 mM MgSO₄ | 1.5 | 0.9 | 0.6 | 210/223/237 | 1.19 | 12.9 nm/8.5% | 14.5 nm/25% |
| 33.3 mM Na₂SO₄ + | 2.0 | 0.9 | 0.9 | 292/195/97 | 1.21 | 14.4 nm/18.3% | 13.6 nm/25.6% |
| 33.3 mM MgCl₂ | | | | | | | |
| 33.3 mM Na₂SO₄ + 25 | 2.0 | 1.0 | 0.8 | 323/180/93 | 1.25 | 13.7 nm/11.7% | 14.5 nm/22.6% |
| mM MgSO₄ | | | | | | | |
| 25 mM MgSO₄ + 100 | 2.0 | 1.0 | 0.6 | 363/184/9 | 1.20 | 14.1 nm/11.9% | 14.4 nm/29.3% |
| mM NaC₂H₃O₂ | | | | | | | |
| 33.3 mM MgCl₂ + 100 | 2.0 | 0.8 | 1.8 | 283/177/17 | 1.18 | 13.8 nm/11.9% | 14.6 nm/32.4% |
| mM NaC₂H₃O₂ | | | | | | | |
| 33.3 mM MgCl₂ + | 2.0 | 0.7 | 1.7 | 287/213/70 | 1.15 | 13.6 nm/11.7% | 16.1 nm/26.1% |
| 100 mM NaCl | | | | | | | |
| 25 mM MgSO₄ + | 2.0 | 0.8 | 1.0 | 287/196/98 | 1.21 | 13.4 nm/11.9% | 15.5 nm/27.6% |
| 100 mM NaCl | | | | | | | |
| 25 mM MgSO₄ + | 2.0 | 1.2 | 0.8 | 339/128/47 | 1.21 | 13.6 nm/11.6% | 15.1 nm/23.3% |
| 33.3 mM MgCl₂ | | | | | | | |

^{a}No Peak 3 was collected. Elution of Peak 1 and Peak 2 occurred at higher ionic strength compared to other salt gradients, and it is unclear whether a Peak 3 would have eluted if the gradient continued to higher ionic strength.
^{b}Further analysis was not performed due to very low observed resolution.

TABLE 4
Dual Salt Screening with Low Concentration Magnesium in the Equilibration/Load Dilution Buffer

| | | | | P1/P2/P3 A₂₈₀ | Peak 2 | | |
|---|---|---|---|---|---|---|---|
| | | | | Integrated | A₂₆₀/ | Peak 2 DLS | Peak 3 DLS |
| Gradient | Initial Mg²⁺ | P1/P2 | P2/P3 | Area | A₂₈₀ | radius (nm)/% | radius (nm)/% |
| Salts | Salt | Resolution | Resolution | (ml* mAU) | ratio | polydispersity | polydispersity |
| 25 mM | 2 mM | 1.0 | 1.1 | 378/196/61 | 1.28 | 13.0 nm/22.8% | 12.2 nm/43.1% |
| MgSO₄ + | MgCl₂ | | | | | | |
| 33.3 mM | | | | | | | |
| MgCl₂ | | | | | | | |
| 25 mM | 1.5 mM | 1.0 | n/a (no | 369/187/none | 1.27 | 12.9 nm/17.0% | None detected |
| MgSO₄ + | MgSO₄ | | Peak 3 | detected | | | |
| 33.3 mM | | | detected) | | | | |
| MgCl₂ | | | | | | | |
| 25 mM | 1.5 mM | 0.9 | 1.0 | 366/221/85 | 1.24 | 13.2 nm/10.3% | 12.0 nm/24.2% |
| MgSO₄ + | MgSO₄ | | | | | | |
| 100 mM | | | | | | | |
| NaC₂H₃O₂ | | | | | | | |
| 33.3 mM | 2 mM | 0.8 | 0.7 | 316/136/20 | 1.21 | 13.4 nm/11.1% | 12.0 nm/19.5% |
| MgCl₂ + | MgCl₂ | | | | | | |
| 100 mM | | | | | | | |
| NaC₂H₃O₂ | | | | | | | |
| 33.3 mM | 1.5 mM | 1.0 | 0.8 | 360/180/75 | 1.30 | 13.0 nm/10.8% | 12.4 nm/20.3% |
| Na₂SO₄ + 25 | MgSO₄ | | | | | | |
| mM MgSO₄ | | | | | | | |

TABLE 5
Load Factor and Scale Comparison for Optimized Elution

| | | | | | | | | | Enrichment | |
|---|---|---|---|---|---|---|---|---|---|---|
| | | | | Gradient | | | | | Factor | |
| | | | Feed | Hold | Peak 2 | | | Eluate % | (Eluate % | Peak 2 DLS |
| | Column | Load Factor | Stream/ | Percent of | A₂₆₀/A₂₈₀ | Vg yield | Cp yield | Full | Full/Feed | radius (nm)/% |
| Run | Size | (cp/mL CV) | % Full | Peak Max | ratio | (%) | (%) | (vg/cp) | % Full) | polydispersity |
| Run A | 0.66 cm ID × | 5 × 10¹³ | FS1/20% | 95% | 1.29 | 62% | 18% | 68% | 3.4 | 12.7 nm/25.02% |
| | 20 cm BH | | Full | | | | | | | |
| Run B | 0.66 cm ID × | 5 × 10¹³ | | 85% | 1.31 | 69% | 22% | 80% | 4 | 12.9 nm/25.47% |
| | 20 cm BH | | | | | | | | | |
| Run C | 0.66 cm ID × | 1.5 × 10¹⁴ | | 95% | 1.30 | 84% | 26% | 74% | 3.7 | 11.5 nm/40.04% |
| | 20 cm BH | | | | | | | | | |
| Run D | 0.66 cm ID × | 1.5 × 10¹⁴ | | 85% | 1.30 | 64% | 21% | 67% | 3.4 | 14.4 nm/11.58% |
| | 20 cm BH | | | | | | | | | |
| Run E | 0.66 cm ID × | 4.5 × 10¹⁴ | | 95% | 1.25 | 91% | 32% | 64% | 3.2 | 13.5 nm/11.41% |
| | 20 cm BH | | | | | | | | | |
| Run F | 3.5 cm ID × | 2 × 10¹³ | | 85% | 1.28 | 80% | 22% | 80% | 4 | 14.1 nm/11.79% |
| | 20 cm BH | | | | | | | | | (N = 2) |
| Run G | 5 cm ID × | 7 × 10¹³ | | 95% | 1.32 | 63% | 24% | 63% | 3.2 | 14.0 nm/14.22% |
| | 20 cm BH | | | | | | | | | |
| Run H | 0.66 cm ID × | 3.9 × 10¹⁴ | FS2/10% | 75% | 1.19 | 64% | 13% | 54% | 5.4 | 14.4 nm/11.53% |
| | 20 cm BH | | Full | | | | | | | |
| Run I | 2.6 cm ID × | 2.0 × 10¹⁴ | | 75% | 1.19 | 60% | 14% | 52% | 5.2 | 15.0 nm/12.38% |
| | 20 cm BH | | | | | | | | | |
| Run J | 2.6 cm ID × | 3.5 × 10¹⁴ | | 75% | 1.24 | 76% | 17% | 51% | 5.1 | 14.5 nm/12.00% |
| | 20 cm BH | | | | | | | | | |
| | Average | | FS1/20% | 85% | 1.30 ± 0.02 | 71% ± 8% | 21% ± 1% | 69% ± 5% | 3.8 ± 0.3 | |
| | | | Full | 95% | 1.29 ± 0.03 | 75% ± 15% | 25% ± 6% | 69% ± 8% | 3.4 ± 0.2 | |
| | | | FS2/10% | 75% | 1.20 ± 0.03 | 67% ± 8% | 15% ± 2% | 52% ± 2% | 5.2 ± 0.2 | |
| | | | Full | | | | | | | |

### Experimental Methods

### Materials

Affinity purified AAV5 load material was produced in-house, with the production process summarized as follows: HEK293 cells were grown in EXP293 media (ThermoFisher, Waltham, MA, USA) with the addition of 4 mM L-Glutamine (ThermoFisher, Waltham, MA, USA) from a stock solution and were triple transfected at a defined ratio of GOI, Helper, and RepCap plasmids. Ninety-six hours post transfection, the harvest was treated with a commercial endonuclease and chemically lysed. The lysate was clarified by two-stage depth filtration, followed by 0.2 μm polishing filtration. The clarified harvest was then concentrated 10-fold across either a 100 kDa single use hollow fiber membrane (Repligen, Waltham, MA, USA) or a 30 kDa flat sheet TFF cassette (Repligen, Waltham, MA, USA) and diafiltered against 5 diavolumes of affinity equilibration buffer. The TFF-1 pool was then frozen at −80° C. until downstream operations were ready to commence. The TFF-1 Pool was thawed and filtered at ambient temperature and affinity purified using Poros AAVX affinity resin (ThermoFisher, Rockford, IL, USA) and the eluate pool was aliquoted and frozen for anion exchange evaluation steps.

NaCl, MgSO4, MgCl2, NaC2H3O2, Na2SO4, sodium phosphate (monobasic), and sodium phosphate (dibasic) were sourced from JT Baker (Radnor, PA, USA). Bis-tris propane was sourced from SAFC (St. Louis, MO, USA). Sucrose was sourced from Pfanstiehl (Waukegan, IL, USA). Poloxamer 188 was sourced from BASF Corporation (Geismar, LA, USA). 0.1 M and 0.5 M sodium hydroxide solutions were sourced from VWR International (Radnor, PA, USA). POROS 50HQ resin, Proteinase K, and Blocker BSA (10×) in TBS were sourced from ThermoFisher (Rockford, IL, USA). 6.6 mm ID×350 mm BH and 35 mm ID×350 mm BH Diba Omnitfit EZ columns (Danbury, CT, USA) and 50 mm ID×450 mm BH Cytiva HiScale columns (Marlbourough, MA, USA) were packed to ˜20 cm with Poros 50HQ resin for this work. All chromatography experiments were performed on either an AKTA Pure 150M or an AKTA Avant (Cytiva, Marlborough, MA, USA). Concentration and diafiltration steps were performed using a Krosflo KR2i TFF system (Repligen, Waltham, MA, USA). Pellicon 3 Cassettes and 0.22 μm Millex-GV PVDF membranes were sourced from MilliporeSigma (Burlington, MA, USA). AAV5 Capsid ELISA kits were sourced from PROGEN Biotechnik GmbH (Heidelberg, Germany). ELISA plate absorbance measurements were determined using a SpectraMax iD5 Multi-Mode Plate Reader (Molecular Devices, Sunnyvale, CA, USA). HL-SAN nuclease for ddPCR was sourced from ArcticZymes (Tromso, Norway). The ddPCR Supermix for Probes, Laemmli buffer, 4-15% SDS-PAGE Gels, blue 520 goat anti-mouse antibody, blue 700 goat anti-rabbit antibody, ChemiDoc MP imager, QX200 Automated Droplet Generator, QX200 Droplet Reader, and C1000 thermal cycler were sourced from Bio-Rad (Hercules, CA, USA). The custom primer probes used for ddPCR were sourced from Integrated DNA Technologies (Coralville, Iowa, USA). For DLS, the DynaPro Plate Reader III was sourced from Wyatt Technology Corporation (Santa Barbara, CA, USA). For the transgene expression assay, HelaRC32 cells were sourced from ATCC (Manassas, VA, USA). The Optima XLA and XLI Analytical Ultracentrifuges were sourced from Beckman Coulter (Brea, CA, USA).

### Anion Exchange Chromatography

For initial linear gradient screening runs, the eluate from affinity chromatography was diluted 10-fold with Buffer A (20 mM bis-tris propane, 0.01% w/v Poloxamer 188, 1% w/v sucrose, pH 9.0) followed by titration to pH 9.0±0.1 using 1 M bis-tris propane. The titrated and diluted eluate was loaded to 2E+13 cp/mL resin on a ˜6.85 mL POROS 50HQ resin packed in a 0.66 cm ID column. The column was sanitized with 0.5 M NaOH, equilibrated with Buffer A, and then eluted with a linear gradient of Buffer B. The AEX eluate was fractionated into 2 CV fixed volume fractions. For the single salt gradient studies, the composition of Buffer B was maintained to be Buffer A composition with the added concentrations of the following salts: 200 mM NaCl, 200 mM NaC2H3O2, 300 mM Na2SO4, 50 mM CaCl2), 50 mM MgCl2, or 100 mM MgSO4.

For the dual salt screening studies, the following concentrations of the respective salts were added to the composition of Buffer A to constitute Buffer B:
33.3 mM Na2SO4, 33.3 mM MgCl2
33.3 mM Na2SO4, 25 mM MgSO4
25 mM MgSO4, 100 mM NaC2H3O2
33.3 mM MgCl2, 100 mM NaC2H3O2
33.3 mM MgCl2, 100 mM NaCl
25 mM MgSO4, 100 mM NaCl
25 mM MgSO4, 33.3 mM MgCl2

The ionic strength gradient for the elution of the single salt and the dual salt screenings are outlined in Tables 3 and 4, respectively. Dual salt runs performed with initial low concentration magnesium salt used the Buffer A composition described above with either 1.5 mM MgSO4 or 2 mM MgCl2 based on condition as dictated in Table 4.

All steps were performed at a flowrate equivalent to a 6-minute residence time (200 cm/hr). The eluate was neutralized to pH 7.0±0.1 using a neutralization buffer (100 mM bis-tris propane, 2 M NaCl, 0.01% w/v Poloxamer 188, pH 6.4) that was pre-loaded in the eluate fractionation vessels, which were subsequently pooled by visual inspection of the chromatogram to obtain representative pools of P1, P2, and P3 for further sampling and analysis. Following elution, the resin was stripped using 5 CV 1 M NaCl, washed with 5 CV water, cleaned with 5 CV 1 M acetic acid, flushed with 5 CV water, base-sanitized with 5 CV 0.5 M NaOH with a 30 min static hold after 3 CV, and stored using 5 CV 0.1 M NaOH. The resolution between the P2 and P1 was determined using the UNICORN 7 Evaluation software default peak resolution algorithm (GE Healthcare, 2014), calculated as follows:

R_s=(V_R2−V_R1)/(2.354*2*(W_h2+W_h1))

where VR1 and Wh1 denote the retention and width at the half height of the previous peak, and VR2 and Wh2 indicate the retention and the width at half height of the current peak.

For runs performed with the optimized gradient mode, the neutralized affinity eluate FS1 material for Runs A-G and FS2 material for Runs H-J was prepared for load as described above to the load factor outlined in Table 3. All steps were performed using a 4 min residence time (300 cm/hr). After pre-use steps, the column was pre-treated with 100% optimized Buffer B from the linear gradient screening runs (20 mM bis-tris propane, 25 mM MgSO4, 35 mM MgCl2, 0.01% w/v Poloxamer 188, 1% w/v sucrose, pH 9.0), followed by equilibration with the selected Buffer A composition (20 mM bis-tris propane, 2 mM MgSO4, 0.01% w/v Poloxamer 188, 1% w/v sucrose, pH 9.0). Runs G-J used a modified Buffer B (20 mM bis-tris propane, 13.5 mM MgSO4, 17.5 mM MgCl2, 0.01% w/v Poloxamer 188, 1% w/v sucrose, pH 9.0). The affinity purified load was diluted 10× in Buffer A, titrated to pH 9.0 with 1 M bis-tris propane, then loaded onto the columns described in Table 5. After completing pre-use, equilibration, load, and wash steps, a hybrid gradient was implemented with an initial linear gradient at 1 mM ionic strength/CV. A 15 CV gradient hold was implemented once the A280 signal reached 85% or 95% of the P1 absorbance maximum as specified in Table 5 to allow for baseline separation of P1 and P2. Pool collection for P1 and P2 targeted 10% of the A280 maximum for P1 and the A260 maximum for P2. Pools were neutralized with 500 mM sodium phosphate, 2 M NaCl, 0.01% w/v Poloxamer 188, 1% w/v sucrose, pH 5.0 to a target of pH 7.0±0.1. After completion of the gradient hold, P2 was eluted with a 7 CV step to 35% B Buffer for Runs A-F, 70% B Buffer for Run G, and 75% B Buffer for Runs H-J. Following elution, the resin was stripped using 5 CV 1 M NaCl, base-sanitized with 5 CV 0.5 M NaOH with a 30 min static hold after 3 CV, and finally stored using 5 CV 0.1 M NaOH.

### Concentration and Diafiltration

The neutralized eluates from the large-scale runs (Runs F and G) were concentrated and diafiltrated (at 6 LMM) with product formulation buffer. A Krosflo KR2i TFF system loaded with 30 kDa NMWCO Pellicon 3 Cassette with Ultracel membrane was utilized for the tangential flow filtration. The final formulated vector product was sterile-filtered with 0.22 μm Millex-GV PVDF membrane filter and stored.

### Vector Genome Concentration Determination by Digital Droplet PCR

Vector titer was determined using digital droplet polymerase chain reaction (ddPCR) described previously (Fu et al., 2019, Hum Gene Ther Methods 30:144-152). AAV5 samples and positive control were first treated with an endonuclease at 37° C. for 30 minutes to remove nucleic acids present outside the capsids. Further, the sample was treated with a Proteinase K digestion step at 55° C. for 30 minutes, followed by 95° C. for 15 minutes to inactivate Proteinase K. Subsequently, the samples and positive control were diluted and combined with ddPCR Supermix for Probes (no dUTP), and custom primer probes targeted against the vector genome sequence and subjected to droplet generation using QX200 Automated Droplet Generator to produce droplets. The droplets plate was subjected to PCR thermal cycling on a C1000 thermal cycler: 95° C. for 10 minutes, 44 cycles of 95° C. for 30 seconds and 60° C. for 100 seconds, 98° C. for 10 minutes, 4° C. for 30 minutes and hold at 4° C. indefinitely. Following the 4° C. 30 minutes, the droplet plate was read on a droplet reader using the FAM/VIC detection. The limit of quantitation (LOQ) for the vector genome titer is 100 copies/reaction.

### AAV5 Capsid Concentration Determination by AAV5 Capsid ELISA

A commercial ELISA kit (Progen, PRAAV5 or PRAAV5XP) was used to quantify the AAV5 Capsid titer. The manufacturer's instructions were followed for reagent preparation and procedure, using 96-well plate strips pre-coated with anti-AAV5 capture antibodies. Briefly, samples were serial diluted in 1× kit buffer prior to plating. Standards and test samples were detected using a biotinylated anti-AAV5 detection antibody and a streptavidin-peroxidase conjugate. Substrate addition generated a colorimetric signal measured at 450 nm with SpectraMax iD5 microplate reader that was directly proportional to the amount of AAV5 capsids in the sample.

### Dynamic Light Scattering

The particle size distribution and the polydispersity of the AAV5 viral vector samples were analyzed using the DynaPro Plate Reader III at 25° C. Each measurement was obtained with 50 μL of neat sample in a 384-well plate using 5 acquisitions lasting 10 seconds each per well. The instrument operated with a laser at 822.8 nm, and the backscattered light was detected at an angle of 150°.

### Empty/Full Capsid Composition by Analytical Ultracentrifugation

The abundance of empty, intermediate, and full AAV capsids is determined by Sedimentation Velocity Analytical Ultracentrifugation (SV-AUC) using a Beckman Coulter Optima XLA or XLI. The assay measures the rate of sedimentation of a molecule in solution in response to an applied centrifugal force. The sedimentation rate provides information on the mass and hydrodynamic shape of the molecular species at a particular concentration within a particular solvent environment. Samples are diluted to a target concentration, centrifuged at 19,000 rpm, analyzed with detection at 230 nm and results were analyzed using SEDFIT continuous C(s) distribution model developed by the National Institutes of Health.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

Unless the context indicates otherwise, it is specifically intended that the various features described herein can be used in any combination.

### NUMBERED EMBODIMENTS

Embodiment 1. A method, comprising:
a) providing a mixture comprising a plurality of recombinant adeno-associated (rAAV) vector particles and one or more impurities, wherein rAAV vector particles of said plurality of rAAV vector particles each include a therapeutic nucleic acid molecule, and wherein said one or more impurities comprise rAAV vector particle aggregates, rAAV vector particles that do not include said therapeutic nucleic acid molecule, and aggregates of rAAV vector particles and rAAV vector particles that do not include said therapeutic nucleic acid molecule;
b) contacting an anion exchange column with said mixture; and
c) contacting said anion exchange column with a first elution buffer and a second elution buffer to provide an eluate, wherein said eluate comprises rAAV vector particles of said plurality of rAAV vector particles,
wherein said first elution buffer comprises magnesium sulfate; and
wherein said second elution buffer comprises magnesium sulfate and magnesium chloride.

Embodiment 2. The method of embodiment 1, wherein the first elution buffer comprises between about 0 millimolar (mM) to about 10 mM magnesium sulfate.

Embodiment 3. The method of embodiment 2, wherein said first elution buffer comprises about 2 mM magnesium sulfate.

Embodiment 4. The method of any one of embodiments 1-3, wherein said second elution buffer comprises between about 10 millimolar (mM) to about 50 mM magnesium sulfate.

Embodiment 5. The method of embodiment 4, wherein said second elution buffer comprises between about 10 mM to about 40 mM magnesium sulfate.

Embodiment 6. The method of any one of embodiments 1-5, wherein said second elution buffer comprises between about 10 millimolar (mM) to about 50 mM magnesium chloride.

Embodiment 7. The method of embodiment 6, wherein said second elution buffer comprises between about 10 mM to about 30 mM magnesium chloride.

Embodiment 8. A method, comprising:
a) providing a mixture comprising a plurality of recombinant adeno-associated (rAAV) vector particles and one or more impurities, wherein rAAV vector particles of said plurality of rAAV vector particles each include a therapeutic nucleic acid molecule, and wherein said one or more impurities comprise rAAV vector particle aggregates, rAAV vector particles that do not include said therapeutic nucleic acid molecule, and aggregates of rAAV vector particles and rAAV vector particles that do not include said therapeutic nucleic acid molecule;
b) contacting an anion exchange column with said mixture; and
c) contacting said anion exchange column with a first elution buffer and a second elution buffer to provide an eluate, wherein said eluate comprises rAAV vector particles of said plurality of rAAV vector particles,
wherein said first elution buffer comprises a sulfate salt; and
wherein said second elution buffer comprises a sulfate salt and a chloride salt.

Embodiment 9. A method, comprising:
a) providing a mixture comprising a plurality of recombinant adeno-associated (rAAV) vector particles and one or more impurities, wherein rAAV vector particles of said plurality of rAAV vector particles each include a therapeutic nucleic acid molecule, and wherein said one or more impurities comprise rAAV vector particle aggregates, rAAV vector particles that do not include said therapeutic nucleic acid molecule, and aggregates of rAAV vector particles and rAAV vector particles that do not include said therapeutic nucleic acid molecule;
b) contacting an anion exchange column with said mixture; and
c) contacting said anion exchange column with a first elution buffer and a second elution buffer to provide an eluate, wherein said eluate comprises rAAV vector particles of said plurality of rAAV vector particles,
wherein said first elution buffer comprises a first salt; and
wherein said second elution buffer comprises a second salt and a third salt, wherein the total salt concentration of said second elution buffer is between about 40-250 millimolar (mM).

Embodiment 10. The method of embodiment 8 or 9, wherein said first elution buffer comprises magnesium sulfate.

Embodiment 11. The method of embodiment 10, wherein the first elution buffer comprises between about 0 millimolar (mM) to about 10 mM magnesium sulfate.

Embodiment 12. The method of embodiment 11, wherein said first elution buffer comprises about 2 mM magnesium sulfate.

Embodiment 13. The method of any one of embodiments 8-12, wherein said second elution buffer comprises magnesium sulfate.

Embodiment 14. The method of embodiment 13, wherein said second elution buffer comprises between about 10 millimolar (mM) to about 50 mM magnesium sulfate.

Embodiment 15. The method of embodiment 14, wherein said second elution buffer comprises between about 10 mM to about 40 mM magnesium sulfate.

Embodiment 16. The method of any one of embodiments 8-15, wherein said second elution buffer comprises magnesium chloride.

Embodiment 17. The method of embodiment 16, wherein said second elution buffer comprises between about 10 millimolar (mM) to about 50 mM magnesium chloride.

Embodiment 18. The method of embodiment 17, wherein said second elution buffer comprises between about 10 mM to about 30 mM magnesium chloride.

Embodiment 19. The method of any one of embodiments 1-18, wherein said first elution buffer and/or said second elution buffer further comprises bis-tris propane.

Embodiment 20. The method of embodiment 19, wherein said first elution buffer and/or said second elution buffer further comprises 10-30 mM bis-tris propane.

Embodiment 21. The method of any one of embodiments 1-20, wherein said first elution buffer and/or said second elution buffer further comprises a poloxamer.

Embodiment 22. The method of embodiment 21, wherein said first elution buffer and/or said second elution buffer further comprises between about 0.001%-0.1% (weight/volume) poloxamer.

Embodiment 23. The method of embodiment 21 or 22, wherein said poloxamer is poloxamer 188.

Embodiment 24. The method of any one of embodiments 1-23, wherein said first elution buffer and/or said second elution buffer further comprises sucrose.

Embodiment 25. The method of embodiment 24, wherein said first elution buffer and/or said second elution buffer further comprises between about 0.1%-5% (weight/volume) sucrose.

Embodiment 26. The method of any one of embodiments 1-25, wherein said first elution buffer and/or said second elution buffer have a pH of about 9.0.

Embodiment 27. The method of any one of embodiments 1-26, wherein c) comprises initially contacting said anion exchange column with an eluant comprising 100% of said first elution buffer and 0% of said second elution buffer and then linearly decreasing the amount of said first elution buffer in said eluant to 0% and linearly increasing the amount of said second elution buffer in said eluant to 100%.

Embodiment 28. The method of any one of embodiments 1-26, wherein c) comprises initially contacting said anion exchange column with an eluant comprising 100% of said first elution buffer and 0% of said second elution buffer and then decreasing the amount of said first elution buffer in said eluant stepwise to 0% and increasing the amount of said second elution buffer in said eluant stepwise to 100%.

Embodiment 29. The method of any one of embodiments 1-26, wherein c) comprises initially contacting said anion exchange column with an eluant comprising 100% of said first elution buffer and 0% of said second elution buffer and then (i) decreasing the amount of said first elution buffer in said eluant to between about 70-90% and increasing the amount of said second elution buffer in said eluant to between about 10-30%; (ii) linearly decreasing the amount of said first elution buffer in said eluant to 0% and linearly increasing the amount of said second elution buffer in said eluant to 100.

Embodiment 30. The method of embodiment 29, wherein c) comprises initially contacting said anion exchange column with an eluant comprising 100% of said first elution buffer and 0% of said second elution buffer and then (i) decreasing the amount of said first elution buffer in said eluant to 80% and increasing the amount of said second elution buffer in said eluant to 20%; (ii) linearly decreasing the amount of said first elution buffer in said eluant to 0% and linearly increasing the amount of said second elution buffer in said eluant to 100.

Embodiment 31. The method of embodiment 29 or 30, wherein during (ii), the amount of said first elution buffer in said eluant and the amount of said second elution buffer in said eluant are held constant over about 7-20 column volumes (CV).

Embodiment 32. The method of embodiment 31, wherein the amount of said first elution buffer in said eluant and the amount of said second elution buffer in said eluant are held constant over about 10 column volumes (CV).

Embodiment 33. The method of embodiment 31 or 32, wherein the amount of said first elution buffer in said eluant is held constant at between about 40-60% and the amount of said second elution buffer in said eluant is held constant at between about 40-60%.

Embodiment 34. The method of any one of embodiments 1-33, wherein said anion exchange column comprises a strong anion exchange resin.

Embodiment 35. The method of embodiment 34, wherein said strong anion exchange resin comprises trialkyl ammonium chloride or hydroxide.

Embodiment 36. The method of embodiment 34, wherein said strong anion exchange resin comprises dialkyl 2-hydroxyethyl ammonium chloride or hydroxide.

Embodiment 37. The method of any one of embodiments 1-33, wherein said anion exchange column is functionalized with quaternary amines.

Embodiment 38. The method of any one of embodiments 1-33, wherein said anion exchange column is functionalized with diethylaminoethanol.

Embodiment 39. The method of any one of embodiment 1-38, further comprising, prior to a),
(i) providing a solution comprising cells and said plurality of rAAV vector particles; (ii) lysing said cells of said solution to produce a lysate; (iii) filtering said lysate to produce a clarified lysate; and (iv) subjecting said clarified lysate to an affinity chromatography step to provide said mixture.

Embodiment 40. The method of embodiment 39, wherein said cells are HEK293 cells.

Embodiment 41. The method of embodiment 39 or 40, wherein said lysate is diluted prior to (iii).

Embodiment 42. The method of any one of embodiments 39-41, wherein said clarified lysate is diluted prior to (iv).

Embodiment 43. The method of any one of embodiments 39-42, further comprising, prior to a), contacting said mixture or a precursor thereof with a cation exchange column.

Embodiment 44. The method of any one of embodiments 1-43, further comprising, prior to a), contacting said mixture or a precursor thereof with a cation exchange column.

Embodiment 45. The method of any one of embodiments 1-44, further comprising, after c), contacting said eluate with a cation exchange column.

Embodiment 46. The method of any one of embodiments 1-45, further comprising, after c), concentrating and/or filtering said rAAV vector particles of said eluate.

Embodiment 47. The method of any one of embodiments 1-46, further comprising, after c), using said rAAV vector particles of said eluate, or a portion thereof, in the preparation of a gene therapy composition.

Embodiment 48. The method of any one of embodiments 1-47, wherein the rAAV of said rAAV vector particles are of serotype AAV1, AAV2, AAV3, AAV4, AAV5, AAV6, AAV7, AAV8, AAV9, AAV10, AAV11, AAV12, AAV13, AAVrh32.33, AAVrh8, AAVrh10, AAVrh74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, or AAV PHP.B.

Embodiment 49. The method of embodiment 48, wherein the rAAV of said rAAV vector particles are of serotype AAV5.

Embodiment 50. The method of any one of embodiments 1-49, wherein the rAAV of said rAAV vector particles is single-stranded rAAV.

Embodiment 51. The method of any one of embodiments 1-50, wherein the rAAV of said rAAV vector particles is self-complementary rAAV.

Embodiment 52. The method of any one of embodiments 1-51, wherein said therapeutic nucleic acid molecule comprises between about 1000-5000 base pairs.

Embodiment 53. The method of embodiment 52, wherein said therapeutic nucleic acid molecule comprises between about 3000-5000 base pairs.

Embodiment 54. The method of any one of embodiments 1-53, wherein said therapeutic nucleic acid molecule comprises a sequence encoding a 21-hydroxylase (21OH) protein.

Embodiment 55. The method of embodiment 54, wherein said sequence is a codon-optimized sequence.

Embodiment 56. The method of any one of embodiments 1-55, wherein said therapeutic nucleic acid molecule is operably linked to a promoter.

Embodiment 57. The method of embodiment 56, wherein said promoter is a cytomegalovirus/0-actin hybrid promoter, PGK promoter, or a promoter specific for expression in an adrenal cortex cell.

Embodiment 58. The method of embodiment 57, wherein said cytomegalovirus/0-actin hybrid promoter is a CAG, CB6, or CBA promoter.

Embodiment 59. The method of any one of embodiments 1-58, wherein said therapeutic nucleic acid molecule comprises at least one AAV inverted terminal repeat (ITR).

Embodiment 60. The method of embodiment 59, wherein said at least one ITR is of serotype AAV2.

Embodiment 61. The method of any one of embodiments 1-60, wherein said therapeutic nucleic acid molecule comprises a Kozak sequence.

Embodiment 62. The method of any one of embodiments 1-61, wherein said therapeutic nucleic acid molecule comprises an miR-122 binding site.

1. A method, comprising:
a) providing a mixture comprising a plurality of recombinant adeno-associated (rAAV) vector particles and one or more impurities, wherein rAAV vector particles of said plurality of rAAV vector particles each include a therapeutic nucleic acid molecule, and wherein said one or more impurities comprise rAAV vector particle aggregates, rAAV vector particles that do not include said therapeutic nucleic acid molecule, and aggregates of rAAV vector particles and rAAV vector particles that do not include said therapeutic nucleic acid molecule;
b) contacting an anion exchange column with said mixture; and
c) contacting said anion exchange column with a first elution buffer and a second elution buffer to provide an eluate, wherein said eluate comprises rAAV vector particles of said plurality of rAAV vector particles,
wherein said first elution buffer comprises magnesium sulfate; and
wherein said second elution buffer comprises magnesium sulfate and magnesium chloride.

2. The method of claim 1, wherein the first elution buffer comprises between about 0 millimolar (mM) to about 10 mM magnesium sulfate.

3. (canceled)

4. The method of claim 1, wherein said second elution buffer comprises between about 10 millimolar (mM) to about 50 mM magnesium sulfate.

5. (canceled)

6. The method of claim 1, wherein said second elution buffer comprises between about 10 millimolar (mM) to about 50 mM magnesium chloride.

7. (canceled)

8. A method, comprising:
a) providing a mixture comprising a plurality of recombinant adeno-associated (rAAV) vector particles and one or more impurities, wherein rAAV vector particles of said plurality of rAAV vector particles each include a therapeutic nucleic acid molecule, and wherein said one or more impurities comprise rAAV vector particle aggregates, rAAV vector particles that do not include said therapeutic nucleic acid molecule, and aggregates of rAAV vector particles and rAAV vector particles that do not include said therapeutic nucleic acid molecule;
b) contacting an anion exchange column with said mixture; and
c) contacting said anion exchange column with a first elution buffer and a second elution buffer to provide an eluate, wherein said eluate comprises rAAV vector particles of said plurality of rAAV vector particles,
wherein said first elution buffer comprises a sulfate salt; and
wherein said second elution buffer comprises a sulfate salt and a chloride salt.

9. A method, comprising:
a) providing a mixture comprising a plurality of recombinant adeno-associated (rAAV) vector particles and one or more impurities, wherein rAAV vector particles of said plurality of rAAV vector particles each include a therapeutic nucleic acid molecule, and wherein said one or more impurities comprise rAAV vector particle aggregates, rAAV vector particles that do not include said therapeutic nucleic acid molecule, and aggregates of rAAV vector particles and rAAV vector particles that do not include said therapeutic nucleic acid molecule;
b) contacting an anion exchange column with said mixture; and
c) contacting said anion exchange column with a first elution buffer and a second elution buffer to provide an eluate, wherein said eluate comprises rAAV vector particles of said plurality of rAAV vector particles,
wherein said first elution buffer comprises a first salt; and
wherein said second elution buffer comprises a second salt and a third salt, wherein the total salt concentration of said second elution buffer is between about 40-250 millimolar (mM).

10. (canceled)

11. The method of claim 9, wherein the first elution buffer comprises between about 0 millimolar (mM) to about 10 mM magnesium sulfate.

12.-13. (canceled)

14. The method of claim 9, wherein said second elution buffer comprises between about 10 millimolar (mM) to about 50 mM magnesium sulfate.

15.-16. (canceled)

17. The method of claim 9, wherein said second elution buffer comprises between about 10 millimolar (mM) to about 50 mM magnesium chloride.

18. (canceled)

19. The method of claim 1, wherein said first elution buffer and/or said second elution buffer further comprises bis-tris propane.

20. (canceled)

21. The method of claim 1, wherein said first elution buffer and/or said second elution buffer further comprises a poloxamer.

22.-23. (canceled)

24. The method of claim 1, wherein said first elution buffer and/or said second elution buffer further comprises sucrose.

25. (canceled)

26. The method of claim 1, wherein said first elution buffer and/or said second elution buffer have a pH of about 9.0.

27. The method of claim 1, wherein c) comprises initially contacting said anion exchange column with an eluant comprising 100% of said first elution buffer and 0% of said second elution buffer and then linearly decreasing the amount of said first elution buffer in said eluant to 0% and linearly increasing the amount of said second elution buffer in said eluant to 100%.

28.-33. (canceled)

34. The method of claim 1, wherein said anion exchange column comprises a strong anion exchange resin.

35.-36. (canceled)

37. The method of claim 1, wherein said anion exchange column is functionalized with quaternary amines.

38. (canceled)

39. The method of claim 1, further comprising, prior to a), (i) providing a solution comprising cells and said plurality of rAAV vector particles; (ii) lysing said cells of said solution to produce a lysate; (iii) filtering said lysate to produce a clarified lysate; and (iv) subjecting said clarified lysate to an affinity chromatography step to provide said mixture.

40.-47. (canceled)

48. The method of claim 1, wherein the rAAV of said rAAV vector particles are of serotype AAV1, AAV2, AAV3, AAV4, AAV5, AAV6, AAV7, AAV8, AAV9, AAV10, AAV11, AAV12, AAV13, AAVrh32.33, AAVrh8, AAVrh10, AAVrh74, AAVhu.68, avian AAV, bovine AAV, canine AAV, equine AAV, ovine AAV, snake AAV, bearded dragon AAV, AAV2i8, AAV2g9, AAV-LK03, AAV7m8, AAV Anc80, or AAV PHP.B.

49. The method of claim 48, wherein the rAAV of said rAAV vector particles are of serotype AAV5.

50.-62. (canceled)
